(19) United States
(12) Patent Application Publication
(10) Pub. No.: US 2024/0156598 A1
(43) Pub. Date: May 16, 2024

(54) SENSING HEART VALVE REPAIR DEVICES

(71) Applicant: EDWARDS LIFESCIENCES CORPORATION, Irvine, CA (US)

(72) Inventors: Daniel James Oliver, Portland, OR (US); Waina Michelle Chu, Tustin, CA (US)

(21) Appl. No.: 18/418,019

(22) Filed: Jan. 19, 2024

Related U.S. Application Data
(63) Continuation of application No. PCT/US2022/037176, filed on Jul. 14, 2022.
(60) Provisional application No. 63/245,731, filed on Sep. 17, 2021.
(60) Provisional application No. 63/223,904, filed on Jul. 20, 2021.

(51) Int. Cl. A61F 2/24; A61M 25/00

(52) U.S. Cl. CPC A61F 2/246; A61F 2/2466; A61M 25/0026; A61F 2220/0091; A61F 2250/0058; A61M 2025/0001

(57) ABSTRACT

A sensing valve repair system includes a delivery system and a heart valve repair device. The delivery system is configured to deploy the heart valve repair device. The sensing valve repair system has a first sensor associated with one or more of the delivery system and the valve repair device. The first sensor is attached to one or more of an inner paddle and a fixed arm of a clasp.

### RELATED APPLICATIONS

The present application is a continuation of PCT application no. PCT/US2022/037176, filed on Jul. 14, 2022, which claims the benefit of U.S. Provisional Application No. 63/245,731 filed on Sep. 17, 2021, titled “Sensing Heart Valve Repair Devices,” and the benefit of U.S. Provisional Application No. 63/223,904 filed on Jul. 20, 2021, titled “Sensing Heart Valve Repair Devices,” which are all incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices or implants in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a prosthetic device or implant within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Sensing valve repair devices or implants and sensing valve repair systems are disclosed herein. The sensing valve repair devices or implants and sensing valve repair systems include one or more sensors. The one or more sensors are configured to sense a characteristic, such as pressure.

A sensing valve repair device includes a valve repair component and one or more sensors. The sensing valve repair device is configured to sense a characteristic, such as pressure, at a proximal end of the valve repair component. The sensing valve repair device is configured to sense a characteristic, such as pressure, at a distal end of the valve repair component.

In some implementations, a sensing valve repair device includes a valve repair component, a first sensor, and a second sensor. The valve repair component has a proximal end and a distal end. The first sensor is connected to the valve repair component and is configured to sense a characteristic at the proximal end of the valve repair component. The second sensor is connected to the valve repair component and is configured to sense a characteristic at the distal end of the valve repair component.

In some examples, a pressure gradient across a native valve (e.g., mitral valve, tricuspid valve, etc.) is determined. A valve repair device can be in the native valve such that a first end of the valve repair device is in communication with blood in an atrium and a second end of the valve repair device is in communication with blood in a ventricle. A pressure of the blood in the atrium is sensed with the valve repair device. A pressure of the blood in the ventricle is sensed with the valve repair device.

In some implementations, an implantable prosthetic device or implant comprises at least a first sensor disposed on the device, wherein the first sensor is configured to determine a proximal pressure, determine a distal pressure, and calculate a pressure gradient based on the proximal pressure and the distal pressure.

In some implementations, a sensing valve repair system includes a delivery system and a heart valve repair device that is delivered by the delivery system. In some implementations, the sensing valve repair system includes first and second sensors. In some implementations, the first and second sensors are associated with and/or part of the delivery system. In some implementations, the first sensor is associated with and/or part of the delivery system and the second sensor is associated with and/or part of the valve repair device. In some implementations, the second sensor is associated with and/or part of the delivery system and the first sensor is associated with and/or part of the valve repair device. The first sensor is configured to sense a characteristic proximal to, or at a proximal end of, the valve repair device, and the second sensor is configured to sense a characteristic distal to, or at a distal end of, the valve repair device.

In some implementations, a sensing valve repair system includes a delivery system, a valve repair device, and first and second sensors. The delivery system includes a steerable catheter, and an implant catheter received inside the steerable catheter. The valve repair device is coupled to the implant catheter. The first sensor is associated with one or more of the delivery catheter, the implant catheter, and the valve repair device. The first sensor is configured to sense a characteristic proximal to, or at a proximal end of, the valve repair device. The second sensor is associated with one or more of the delivery system and the valve repair device. The second sensor is configured to sense a characteristic distal to, or at a distal end of, the valve repair device.

A method of sensing a pressure gradient across a native valve is disclosed. In some implementations, the method includes using a delivery system to implant a valve repair device in the native valve. One or more components of the delivery system and a first end of the valve repair device are in communication with blood in an atrium. At least one of a component of the delivery system and a second end of the valve repair device is in communication with blood in a ventricle. Pressure of the blood in the atrium is sensed with a component of the delivery system in communication with blood in an atrium and/or the first end of the valve repair device. Pressure of the blood in the ventricle is sensed a with a component of the delivery system in communication with blood in the ventricle and/or the second end of the valve repair device.

In some implementations, the valve repair device can have a first sensor at the first end of the valve repair device and the valve repair device can have a second sensor at the second end of the valve repair device. The pressure of the blood in the atrium and the pressure of the blood in the ventricle can be transmitted. A gradient between the pressure of the blood in the atrium and the pressure of the blood in the ventricle can be transmitted. The sensed pressure in the atrium can be stored and the sensed pressure in the ventricle can be stored. A flow rate based on the pressure of the blood in the atrium and the pressure of the blood in the ventricle can be transmitted. A heart rate based on the pressure of the blood in the atrium and the pressure of the blood in the ventricle can be determined.

The above method(s) can be performed on a living animal or on a simulation, such as on a cadaver, cadaver heart, simulator (e.g., with simulated body parts, heart, tissue, etc.), etc.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples of the present disclosure, a more particular description of the certain examples will be made by reference to various aspects of the appended drawings. It is appreciated that these drawings depict only typical examples of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a front view of the implantable device or implant of FIG. 22;

FIG. 24 shows a side view of the implantable device or implant of FIG. 22;

FIG. 25 shows a front view of the implantable device or implant of FIG. 22 with a cover covering the paddles and a coaptation element or spacer;

FIG. 26 shows a top perspective view of the implantable device or implant of FIG. 22 in an open position;

FIG. 27 shows a bottom perspective view of the implantable device or implant of FIG. 22 in an open position;

FIG. 28 shows a clasp for use in an implantable device or implant;

FIG. 29 shows a portion of native valve tissue grasped by a clasp;

FIG. 30 shows a side view of an example implantable device or implant in a partially-open position with clasps in a closed position;

FIG. 31 shows a side view of an example implantable device or implant in a partially-open position with clasps in an open position;

FIG. 32 shows a side view of an example implantable device or implant in a half-open position with clasps in a closed position;

FIG. 33 shows a side view of an example implantable device or implant in a half-open position with clasps in an open position;

FIG. 34 shows a side view of an example implantable device or implant in a three-quarters-open position with clasps in a closed position;

FIG. 35 shows a side view of an example implantable device or implant in a three-quarters-open position with clasps in an open position;

FIG. 36 shows a side view of an example implantable device in a fully open or full bailout position with clasps in a closed position;

FIG. 37 shows a side view of an example implantable device in a fully open or full bailout position with clasps in an open position;

FIGS. 38-49 show the example implantable device or implant of FIGS. 30-38, including a cover, being delivered and implanted within a native valve;

FIG. 50 is a schematic view illustrating a path of native valve leaflets along each side of a coaptation element or spacer of an example valve repair device or implant;

FIG. 51 is a top schematic view illustrating a path of native valve leaflets around a coaptation element or spacer of an example valve repair device or implant;

FIG. 52 illustrates a coaptation element or spacer in a gap of a native valve as viewed from an atrial side of the native valve;

FIG. 53 illustrates a valve repair device or implant attached to native valve leaflets with the coaptation element or spacer in the gap of the native valve as viewed from a ventricular side of the native valve;

FIG. 54 is a perspective view of a valve repair device or implant attached to native valve leaflets with the coaptation element or spacer in the gap of the native valve shown from a ventricular side of the native valve;

FIG. 55 shows a perspective view of an example implantable device or implant in a closed position;

FIG. 56 shows a perspective view of an example clasp of an example implantable device or implant in a closed position;

FIG. 57 illustrates a valve repair device with paddles in an open position;

FIG. 58 illustrates the valve repair device of FIG. 57, in which the paddles are in the open position and gripping members are moved to create a wider gap between the gripping members and paddles;

FIG. 59 illustrates the valve repair device of FIG. 57, in which the valve repair device is in the position shown in FIG. 7 with valve tissue placed between the gripping members and the paddles;

FIG. 60 illustrates the valve repair device of FIG. 57, in which the gripping members are moved to lessen the gap between the gripping members and the paddles;

FIGS. 61A-61B illustrate the movement of the paddles of the valve repair device of FIG. 57 from the open position to a closed position;

FIG. 62 illustrates the valve repair device of FIG. 57 in a closed position, in which the gripping members are engaging valve tissue;

FIG. 63 illustrates the valve repair device of FIG. 57 after being disconnected from a delivery device and attached to valve tissue, in which the valve repair device is in a closed and locked condition;

FIG. 64 shows an example implantable device or implant and associated sensor(s) implanted in a native valve;

FIG. 65 shows an example implantable device or implant and associated sensor(s) implanted in the native valve;

FIG. 66 shows an example implantable device or implant and associated sensor(s) implanted in the native valve;

FIG. 67 shows an example implantable device or implant and associated sensor(s) implanted in the native valve;

FIG. 68 shows a perspective view of an example implantable device or implant and associated sensor(s) implanted in the native valve;

FIG. 69 shows a perspective view of an example implantable device or implant and associated sensor(s);

FIG. 70 shows a perspective view of an example implantable device or implant and associated sensor(s).

FIG. 71 shows a perspective view of an example implantable device or implant and associated sensor(s).

FIG. 72 shows a perspective view of an example implantable device or implant and associated sensor(s).

FIG. 73 shows a perspective view of an example implantable device or implant and associated sensor(s).

FIG. 74 shows a perspective view of an example implantable device or implant and associated sensor(s).

FIG. 75 shows a perspective view of an example implantable device or implant and associated sensor(s).

FIG. 76 shows a perspective view of an example implantable device or implant and associated sensor(s).

FIG. 77 shows a perspective view of an example implantable device or implant and associated sensor(s).

FIG. 78 shows an example valve repair system and associated sensor(s).

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of implantable devices, valve repair devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

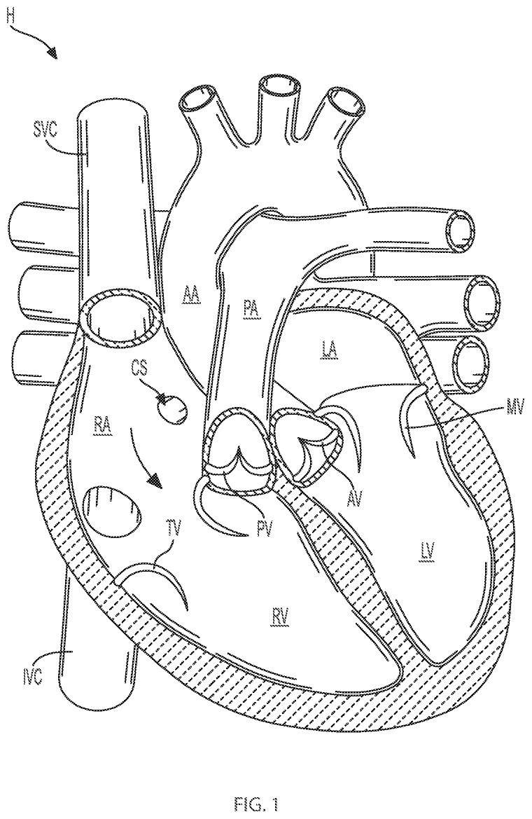

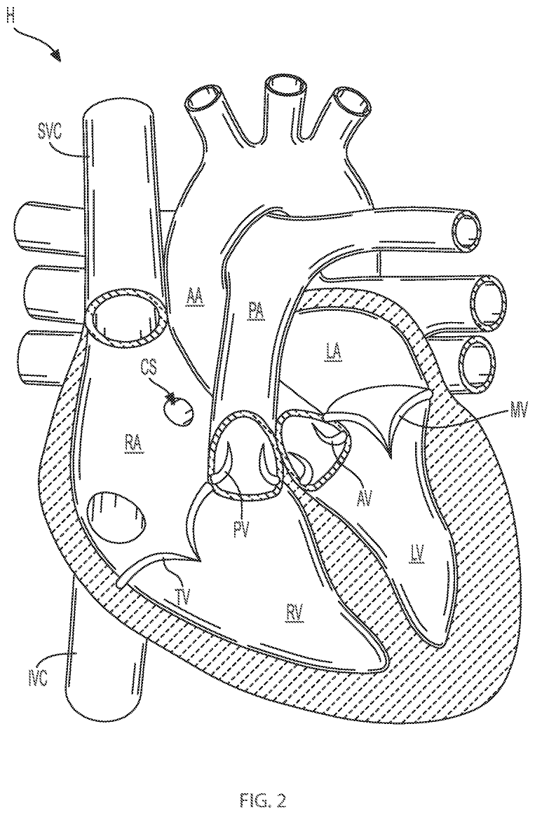

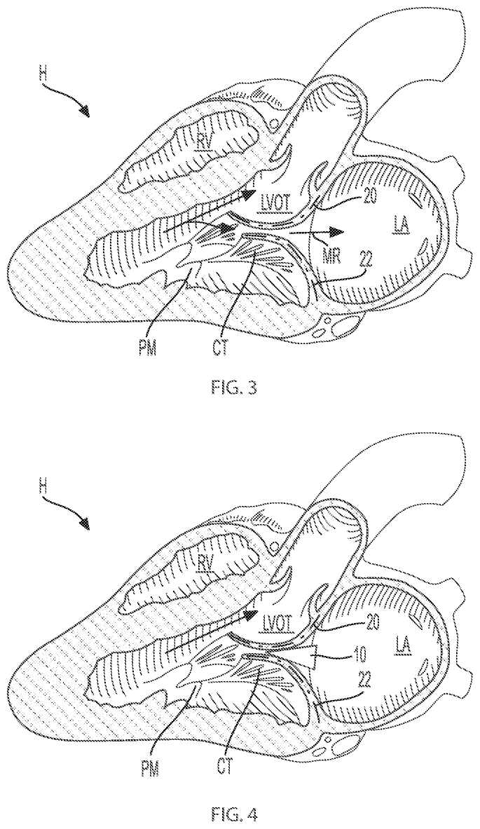

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent or inhibit blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

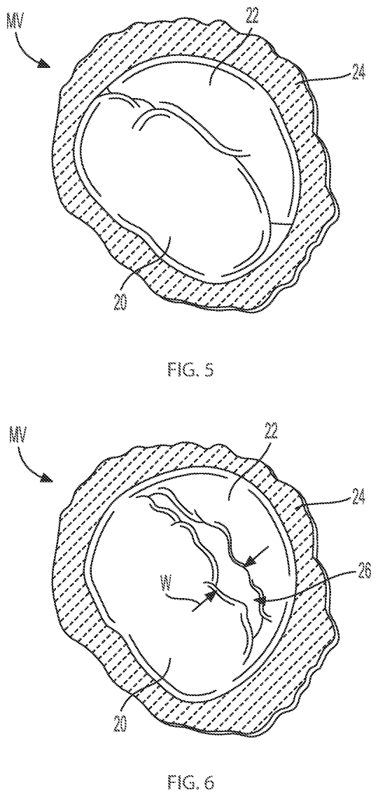

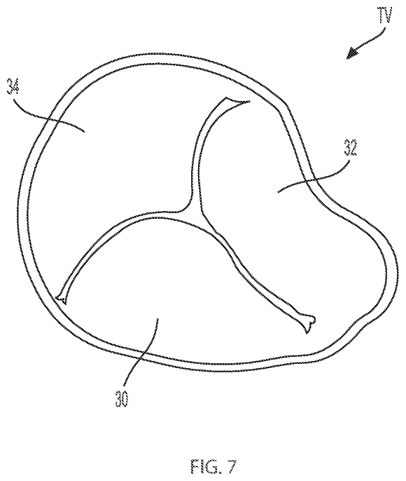

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, spacer, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, prevents the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device (e.g., implantable prosthetic device, etc.) or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing or preventing regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaption element, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

Referring now to FIGS. 8-15, a schematically illustrated implantable device or implant 100 (e.g., a prosthetic spacer device, valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system or other means for delivery 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 or means for coapting (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the means for actuating or actuation element 112 opens and closes the anchor portion 106 of the device 100 to grasp the native valve leaflets during implantation. The means for actuating or actuation element 112 (as well as other means for actuating and actuation elements herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the means for coapting or coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and means for actuating or actuation element 112 (e.g., actuation wire, actuation shaft, etc.). These can be configured to extend through a guide catheter/sheath (e.g., a transseptal sheath, etc.). In some implementations, the means for actuating or actuation element 112 extends through a delivery catheter and the means for coapting or coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the means for actuating or actuation element 112 slides through the collar or other attachment element and, in some implementations, through a means for coapting or coaptation element 110 during actuation to open and close the paddles 120, 122 of the anchor portion 106 and/or anchors 108.

In some implementation, the anchor portion 106 and/or anchors 108 can include attachment portions or gripping members. The illustrated gripping members can comprise clasps 130 that include a base or fixed arm 132, a moveable arm 134, optional barbs, friction-enhancing elements, or other means for securing 136 (e.g., protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate means for coapting or coaptation element 110. In some implementations, the clasps (e.g., barbed clasps, etc.) have flat surfaces and do not fit in a recess of the inner paddle. Rather, the flat portions of the clasps are disposed against the surface of the inner paddle 122. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs, friction-enhancing elements, or means for securing 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. This pinching force remains constant regardless of the position of the inner paddles 122. Optional barbs, friction-enhancing elements, or other means for securing 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a means for coapting or coaptation element 110. The clasps 130 can be used to grasp and/or further secure the native leaflets by engaging the leaflets with optional barbs, friction-enhancing elements, or means for securing 136 and pinching the leaflets between the moveable and fixed arms 134, 132. The optional barbs, friction-enhancing elements, or other means for securing 136 (e.g., barbs, protrusions, ridges, grooves, textured surfaces, adhesive, etc.) of the clasps or barbed clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

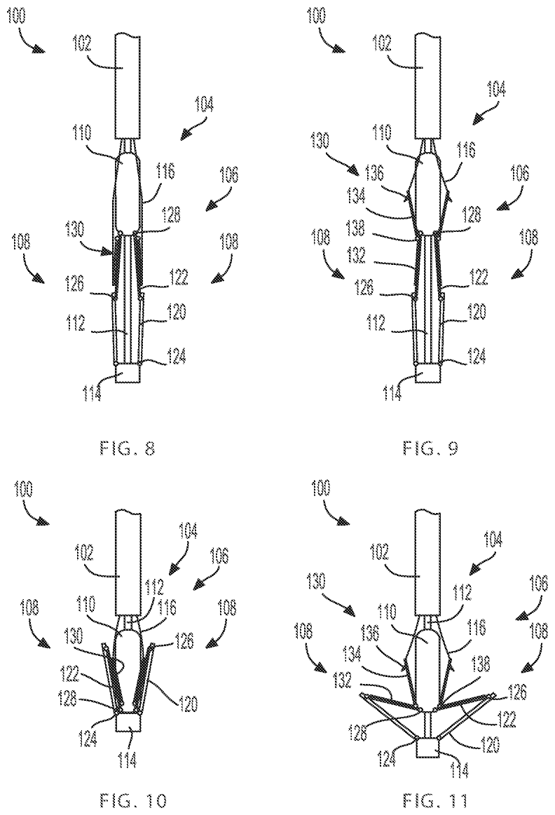

Referring now to FIG. 8, the device 100 is shown in an elongated or fully open condition for deployment from an implant delivery catheter of the delivery system 102. The device 100 is disposed at the end of the catheter of the delivery system 102 in the fully open position, because the fully open position takes up the least space and allows the smallest catheter to be used (or the largest device 100 to be used for a given catheter size). In the elongated condition the cap 114 is spaced apart from the means for coapting or coaptation element 110 such that the paddles 120, 122 are fully extended. In some implementations, an angle formed between the interior of the outer and inner paddles 120, 122 is approximately 180 degrees. The clasps 130 are kept in a closed condition during deployment through the delivery system 102 so that the optional barbs, friction-enhancing elements, or other means for securing 136 (FIG. 9) do not catch or damage the delivery system 102 or tissue in the patient's heart.

Referring now to FIG. 9, the device 100 is shown in an elongated detangling condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the means for actuating or actuation element 112 is retracted to pull the cap 114 towards the means for coapting or coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the means for coapting or coaptation element 110 cause the paddles or gripping elements to move radially outward. During movement from the open to closed position, the outer paddles 120 maintain an acute angle with the means for actuating or actuation element 112. The outer paddles 120 can optionally be biased toward a closed position. The inner paddles 122 during the same motion move through a considerably larger angle as they are oriented away from the means for coapting or coaptation element 110 in the open condition and collapse along the sides of the means for coapting or coaptation element 110 in the closed condition. In some implementations, the inner paddles 122 are thinner and/or narrower than the outer paddles 120, and the connection portions 126, 128 (e.g., joints, flexible connections, etc.) connected to the inner paddles 122 can be thinner and/or more flexible. For example, this increased flexibility can allow more movement than the connection portion 124 connecting the outer paddle 120 to the cap 114. In some implementations, the outer paddles 120 are narrower than the inner paddles 122. The connection portions 126, 128 connected to the inner paddles 122 can be more flexible, for example, to allow more movement than the connection portion 124 connecting the outer paddle 120 to the cap 114. In some implementations, the inner paddles 122 can be the same or substantially the same width as the outer paddles.

Referring now to FIGS. 11-13, the device 100 is shown in a partially open, grasp-ready condition. To transition from the fully closed to the partially open condition, the means for actuating or actuation element (e.g., actuation wire, actuation shaft, etc.) is extended to push the cap 114 away from the means for coapting or coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single means for actuating or single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. For example, the device 100 can have two actuation elements and two independent caps (or other attachment portions), such that one independent actuation element (e.g., wire, shaft, etc.) and cap (or other attachment portion) are used to control one paddle, and the other independent actuation element and cap (or other attachment portion) are used to control the other paddle.

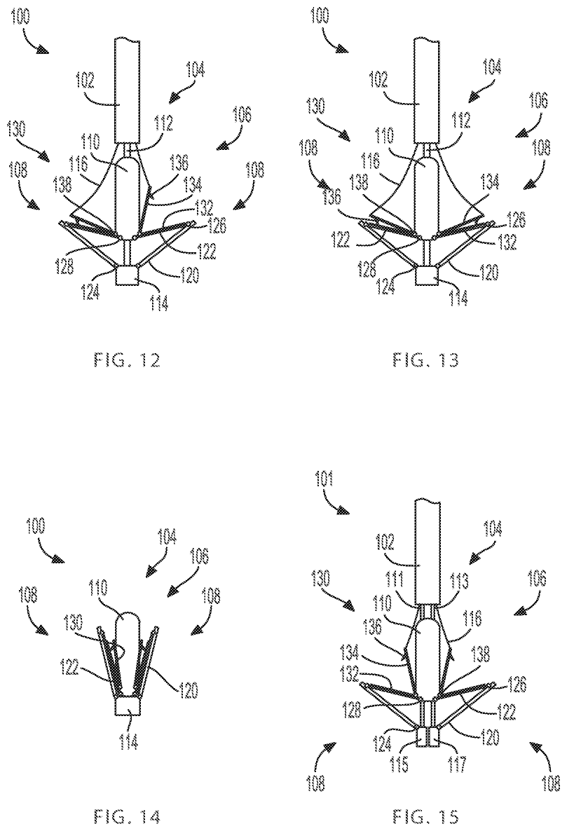

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system or means for delivery 102 and means for actuating or actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the means for coapting or coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 101 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the means for actuating or actuation element 111 is extended to push the cap 115 away from the means for coapting or coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the means for actuating or actuation element 113 is extended to push the cap 115 away from the means for coapting or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single means for actuating or actuation element 112.

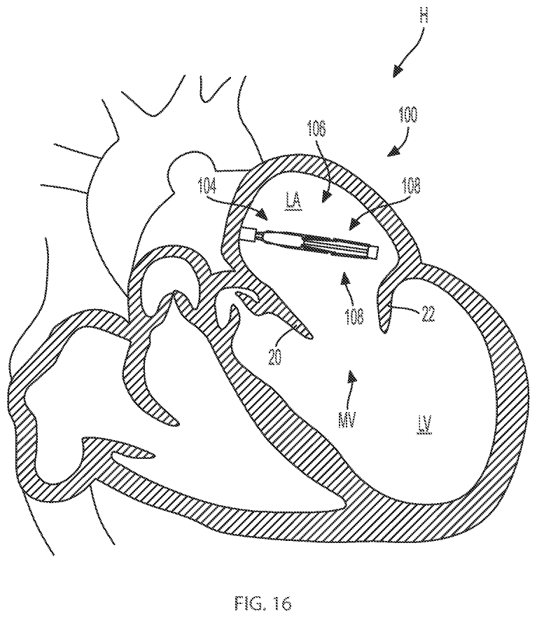

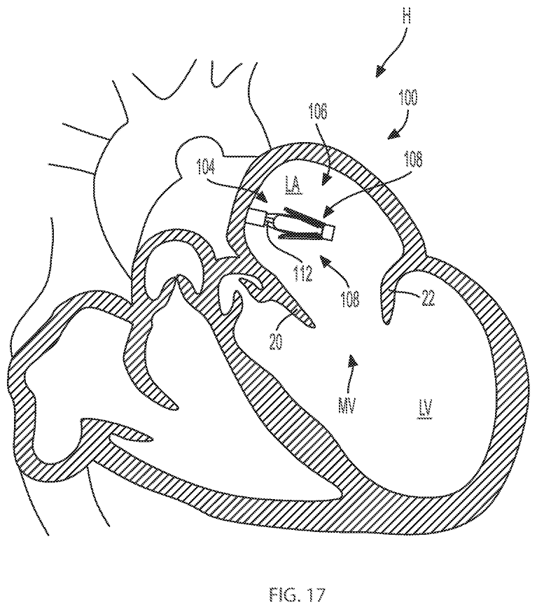

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The means for actuating or actuation element 112 is then retracted to move the implant/device into the fully closed condition shown in FIG. 17.

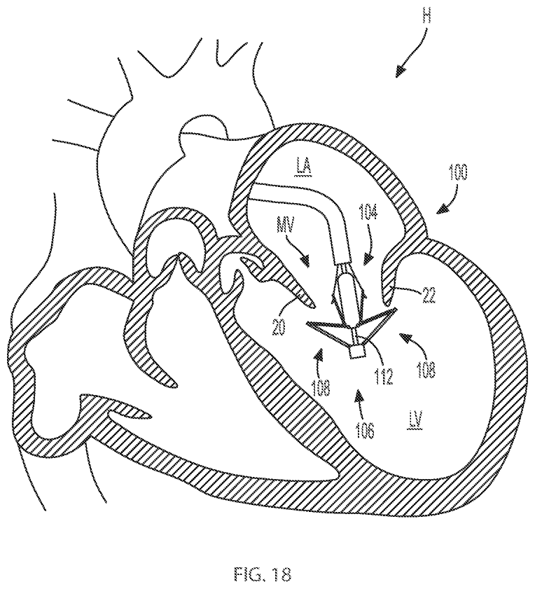

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

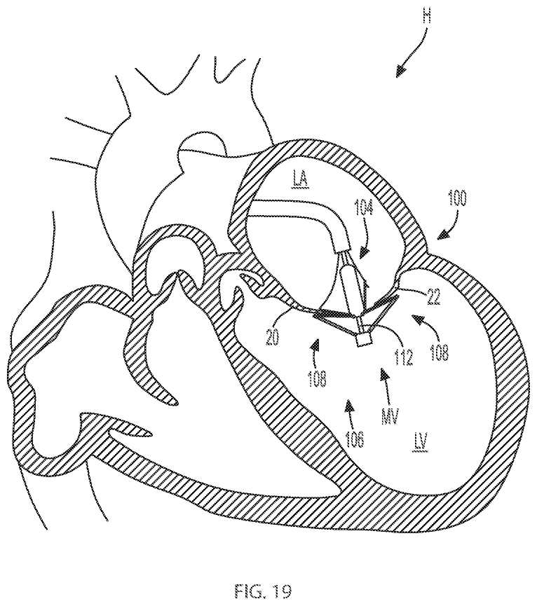

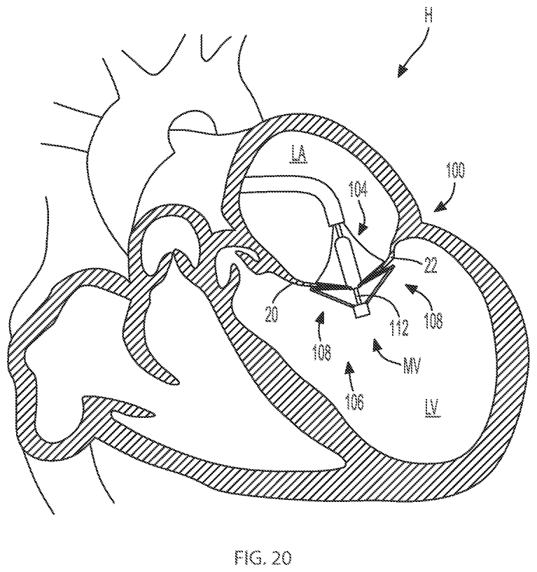

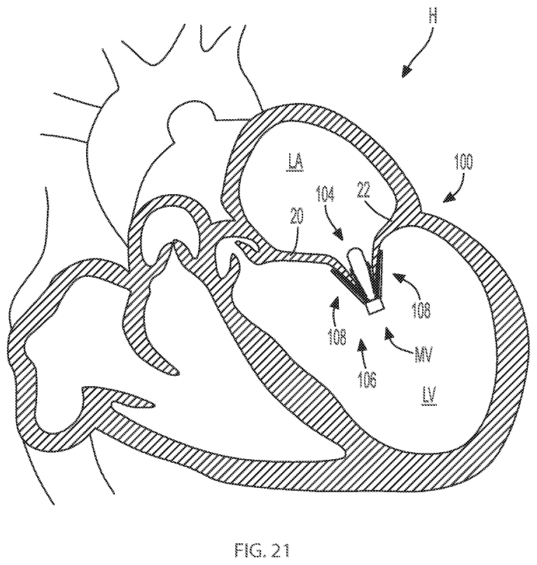

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), means for actuating or actuation element 112 and actuation lines 116 are then retracted and the device or implant 100 is fully closed and deployed in the native mitral valve MV.

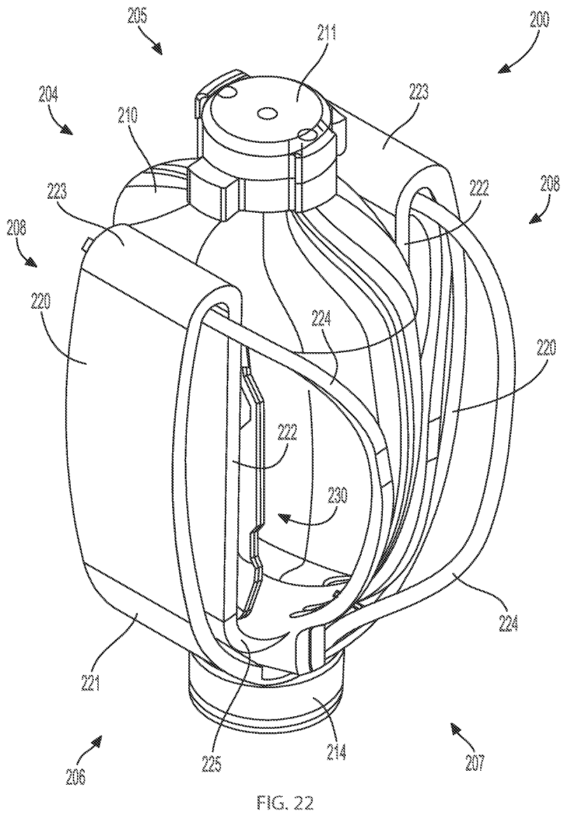

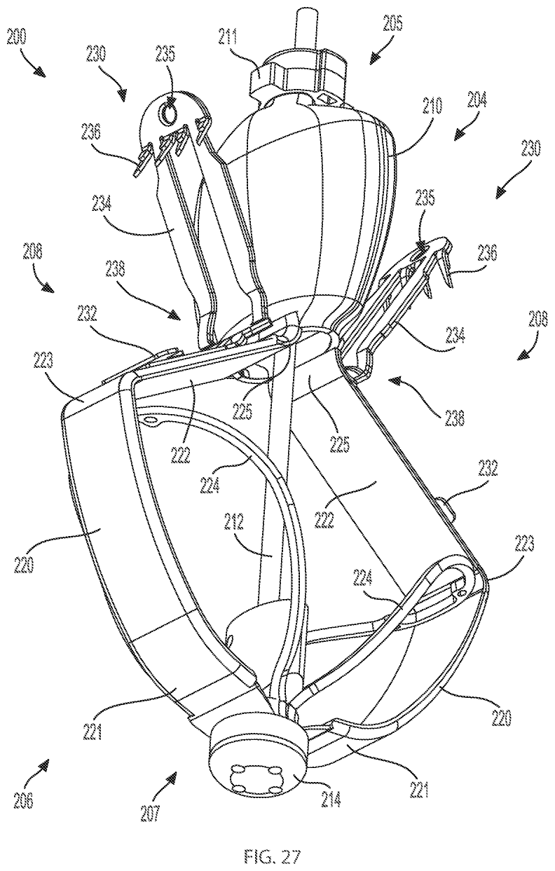

Referring now to FIGS. 22-27, an example of an implantable device or implant or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

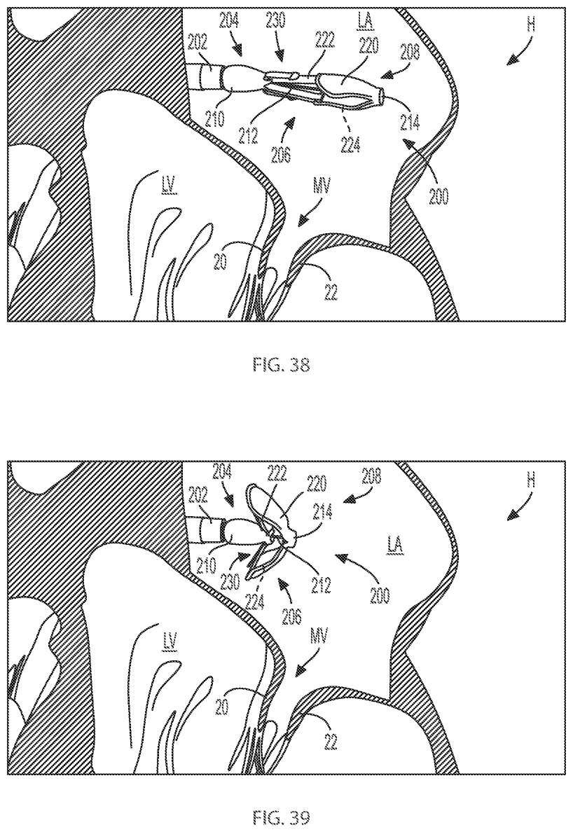

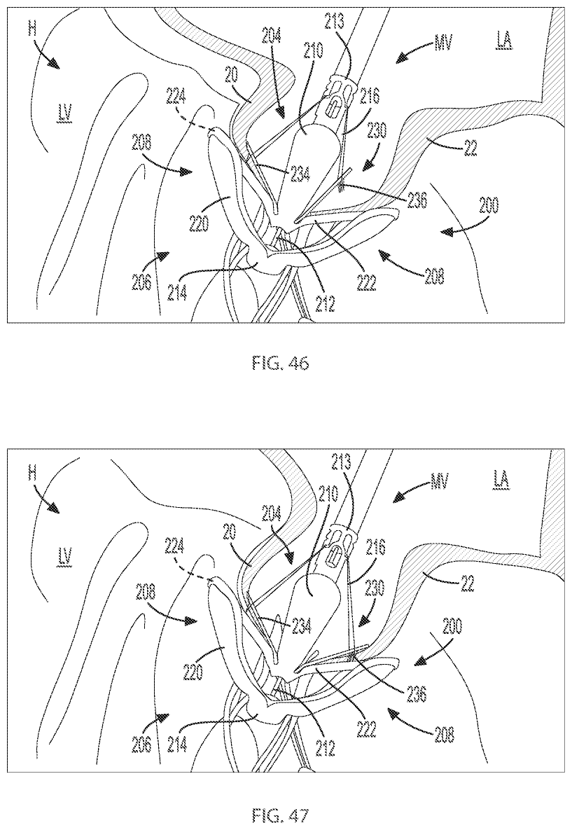

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism 213 (FIGS. 43-49) of a delivery system 202 (FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the implantable device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

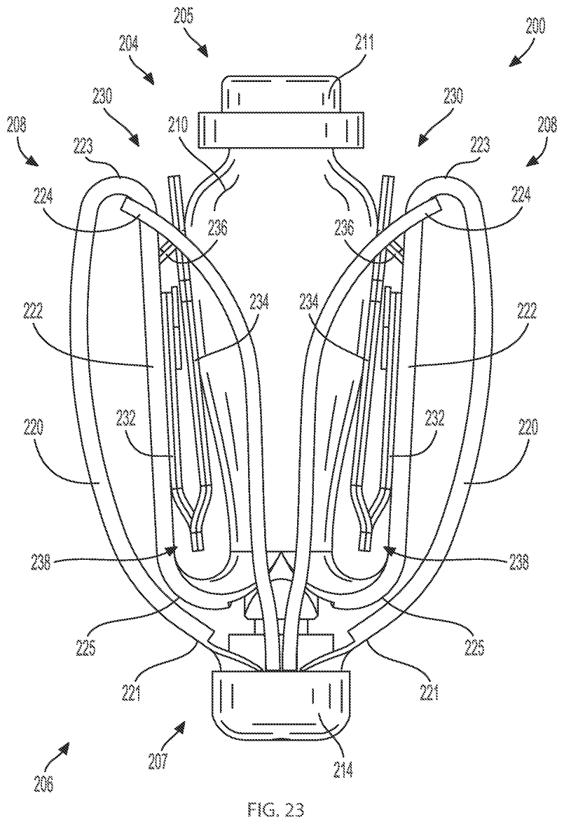

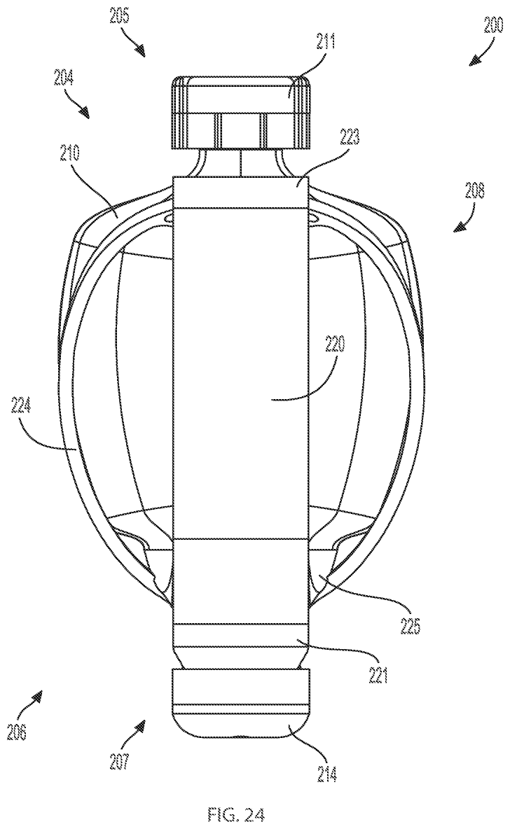

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the implantable device or implant 200, such that the native leaflets coapt entirely around the coaptation member or coaptation element 210. This can, for example, improve sealing of the native leaflets 20, 22 and thus prevent or further reduce mitral regurgitation.

In some implementations the clasps comprise a moveable arm coupled to the anchors. In some implementations, the clasps 230 include a base or fixed arm 232, a moveable arm 234, optional barbs 236, and a joint portion 238. The fixed arms 232 are attached to the inner paddles 222, with the joint portion 238 disposed proximate the coaptation element 210. The joint portion 238 is spring-loaded so that the fixed and moveable arms 232, 234 are biased toward each other when the clasp 230 is in a closed condition. In some implementations, the clasps 230 include friction-enhancing elements or means for securing, such as optional barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the moveable arms 234 are opened to open the clasps 230 and expose the optional barbs or other friction-enhancing elements 236. The clasps 230 are opened by applying tension to actuation lines 216 (e.g., as shown in FIGS. 43-48) attached to holes 235 in the moveable arms 234, thereby causing the moveable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the moveable and fixed arms 234, 232 of the clasp 230. The tissue of the leaflet 20, 22 is not pierced by the optional barbs or friction-enhancing elements 236, though in some implementations the optional barbs 236 can partially or fully pierce through the leaflet 20, 22. The angle and height of the optional barbs or friction-enhancing elements 236 relative to the moveable arm 234 helps to secure the leaflet 20, 22 within the clasp 230. In particular, a force pulling the implant off of the native leaflet 20, 22 will encourage the optional barbs or friction-enhancing elements 236 to further engage the tissue, thereby ensuring better retention. Retention of the leaflet 20, 22 in the clasp 230 is further improved by the position of fixed arm 232 near the optional barbs/friction-enhancing elements 236 when the clasp 230 is closed. In this arrangement, the tissue is formed by the fixed arms 232 and the moveable arms 234 and the optional barbs/friction-enhancing elements 236 into an S-shaped torturous path. Thus, forces pulling the leaflet 20, 22 away from the clasp 230 will encourage the tissue to further engage the optional barbs/friction-enhancing elements 236 before the leaflets 20, 22 can escape. For example, leaflet tension during diastole can encourage the optional barbs 236 to pull toward the end portion of the leaflet 20, 22. Thus, the S-shaped path can utilize the leaflet tension during diastole to more tightly engage the leaflets 20, 22 with the optional barbs/friction-enhancing elements 236.

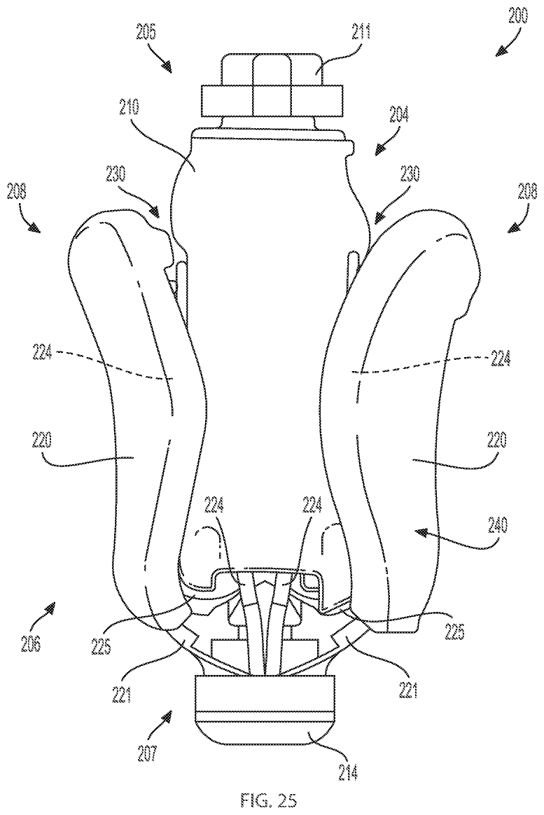

Referring to FIG. 25, the prosthetic device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the prosthetic device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the implantable prosthetic device or implant 200.

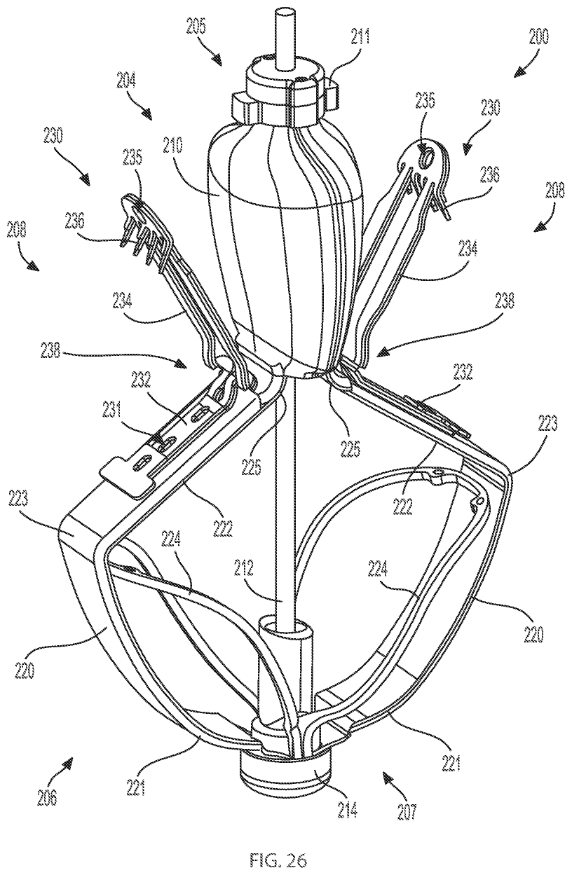

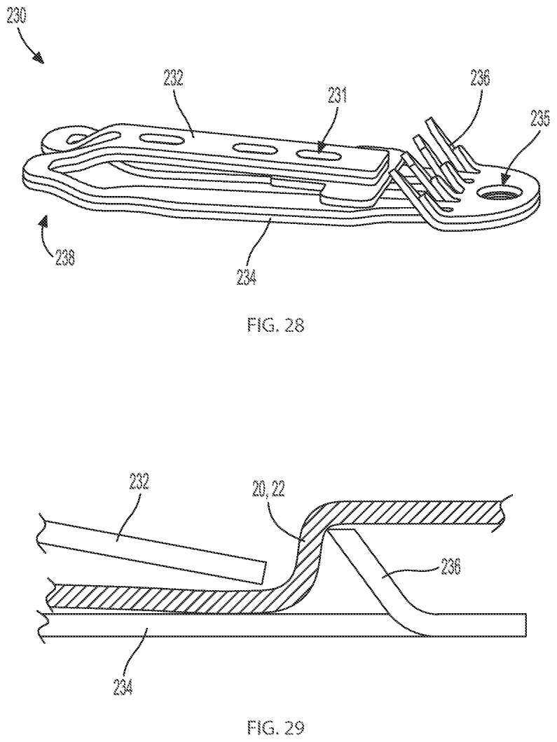

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 100 illustrated in FIG. 15).

In some implementations, the clasps 230 further secure the native leaflets 20, 22 by engaging the leaflets 20, 22 with optional barbs and/or other friction-enhancing elements 236 and pinching the leaflets 20, 22 between the moveable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to more securely grip the leaflets 20, 22 when the device 200 is closed (e.g., as can be seen in FIG. 51). The curved shape and rounded edges of the outer paddle 220 also prohibits or inhibits tearing of the leaflet tissue.

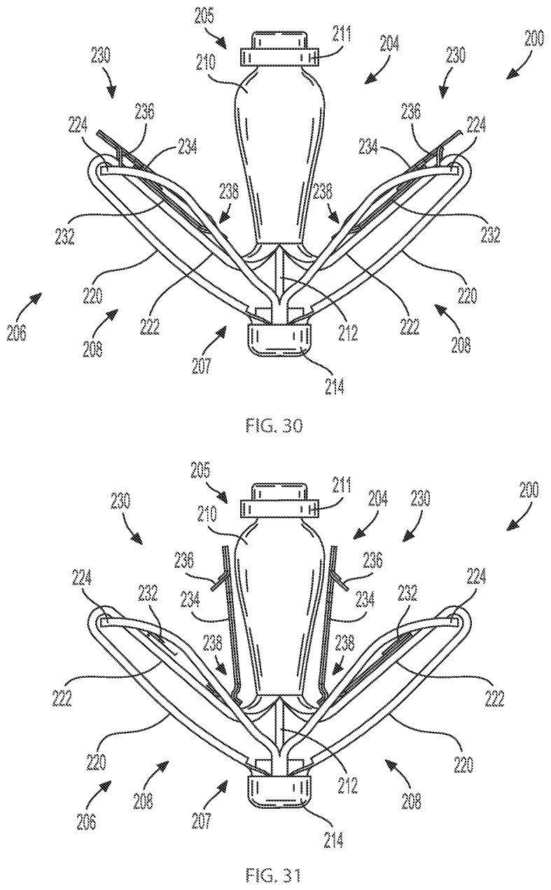

Referring now to FIGS. 30-37, the implantable device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and moveable arms 232, 234 of the clasp 230 is limited to the extent that the inner paddle 222 has spread away from the coaptation element 210.

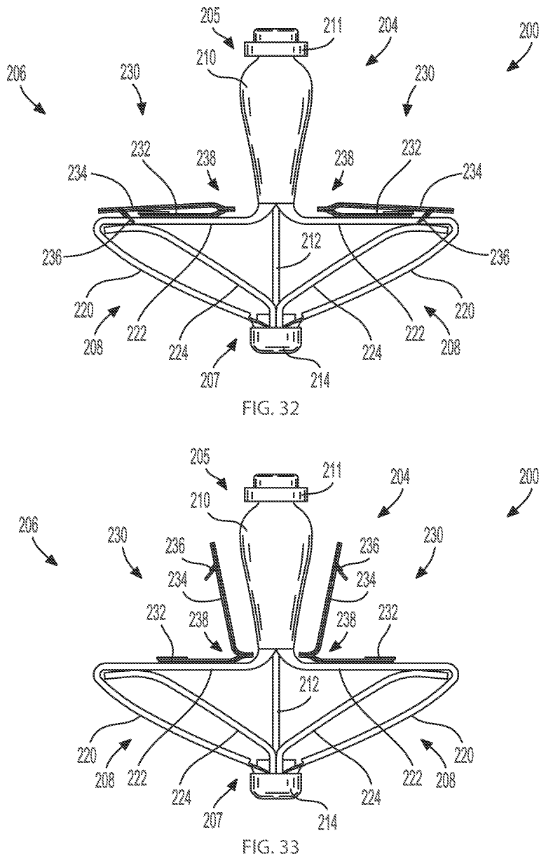

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and moveable arms 232, 234.

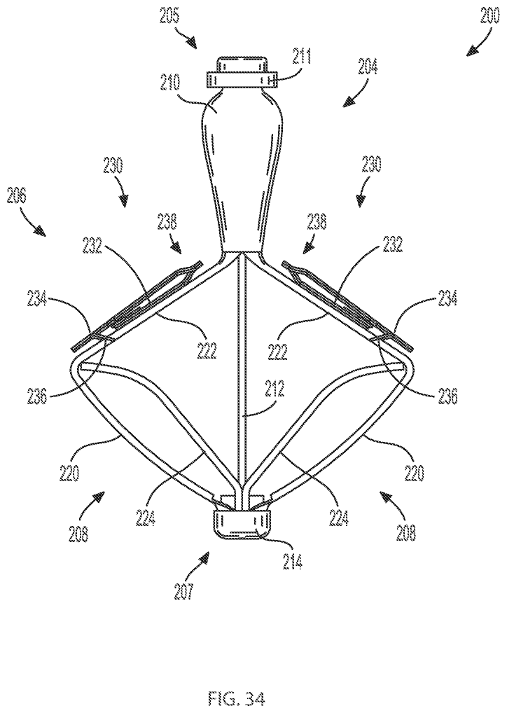

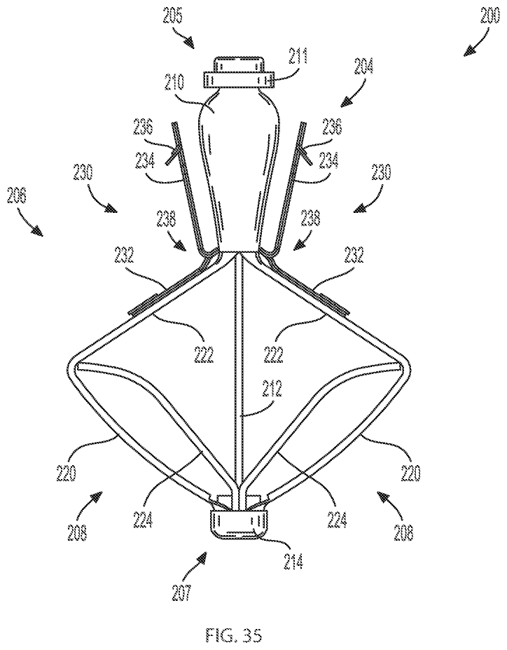

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and moveable arms 232, 234.

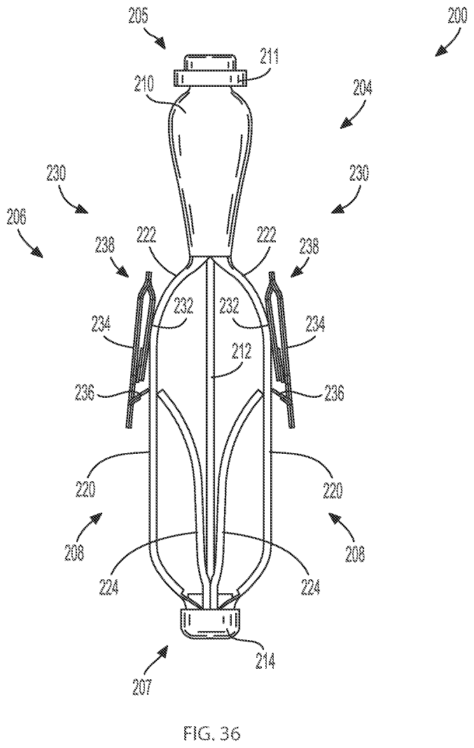

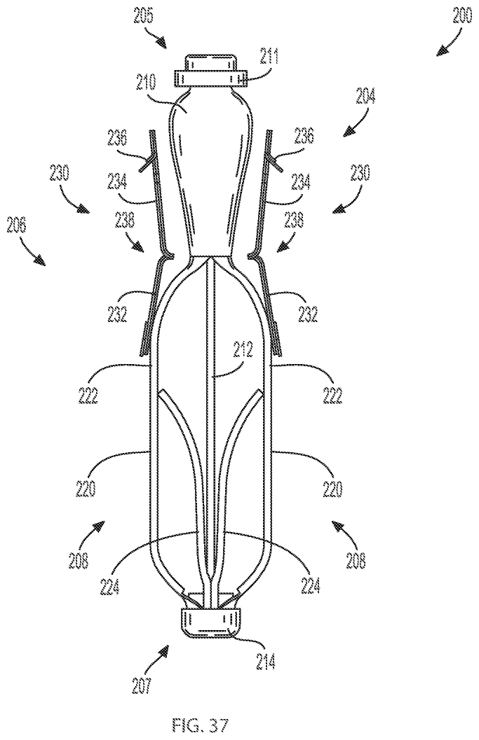

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and moveable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the prosthetic device or implant 200 such that the anchors 208 can extend to a straight or approximately straight configuration (e.g., approximately 120-180 degrees relative to the coaptation element 210) can provide several advantages. For example, this configuration can reduce the radial crimp profile of the prosthetic device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the prosthetic device or implant 200 will become entangled in native anatomy (e.g., chordae tendineae CT shown in FIGS. 3 and 4) when positioning and/or retrieving the prosthetic device or implant 200 into the delivery system 202.

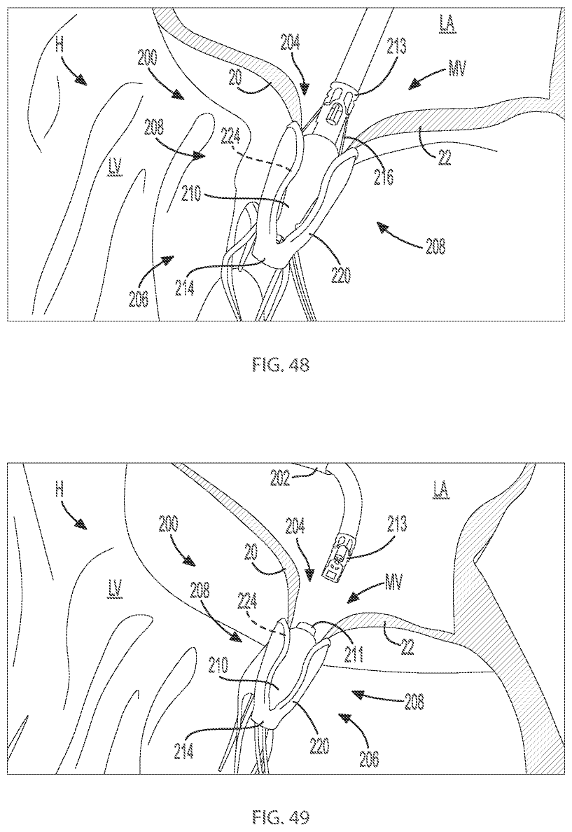

Referring now to FIGS. 38-49, an example implantable device 200 is shown being delivered and implanted within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

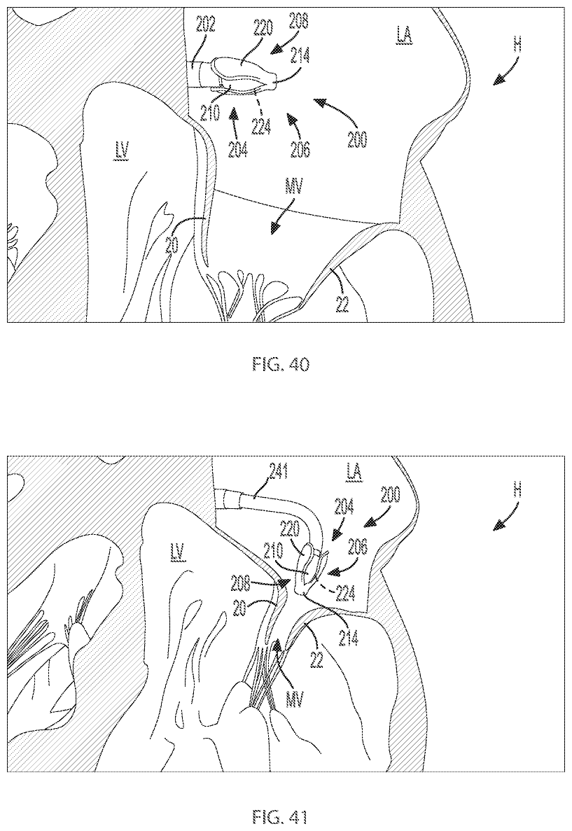

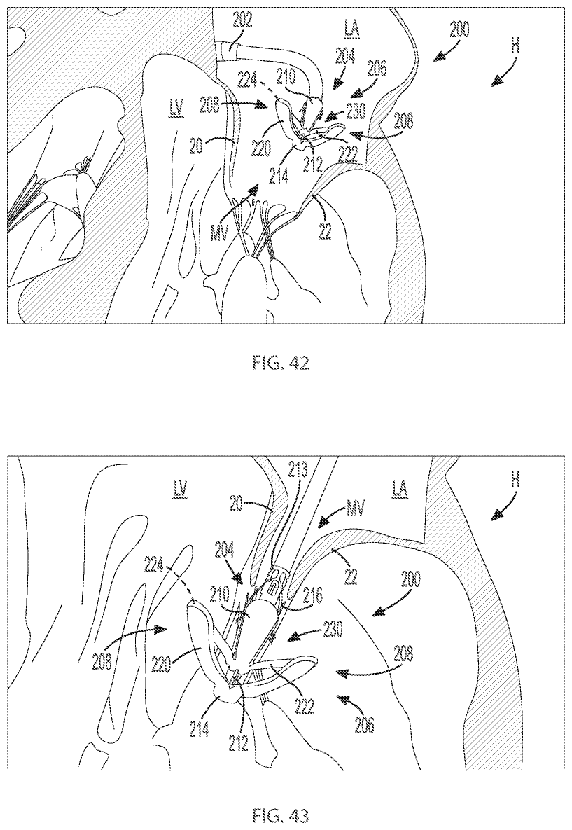

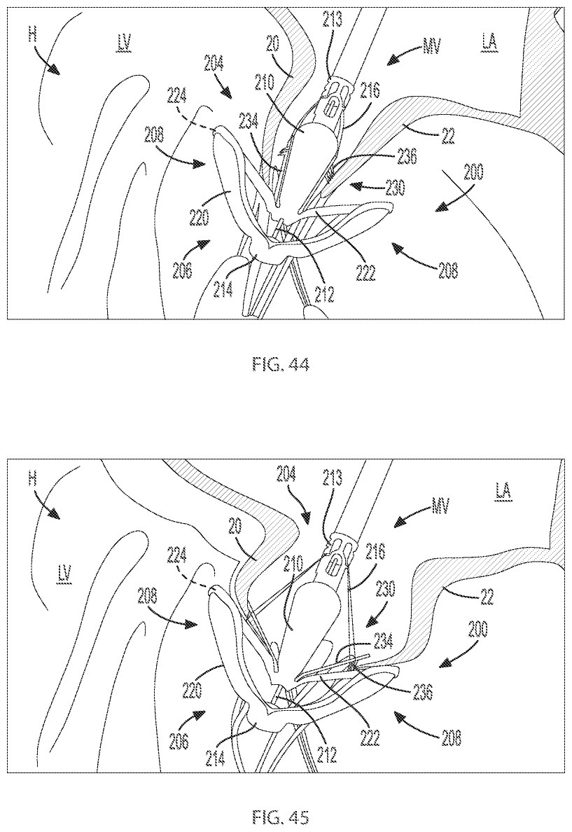

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy— produced in a wire, sheet, tubing, or laser sintered powder—and are biased to hold the outer paddles 220 closed around the inner paddles 222, coaptation element 210, and/or the clasps 230 pinched around native leaflets 20, 22.

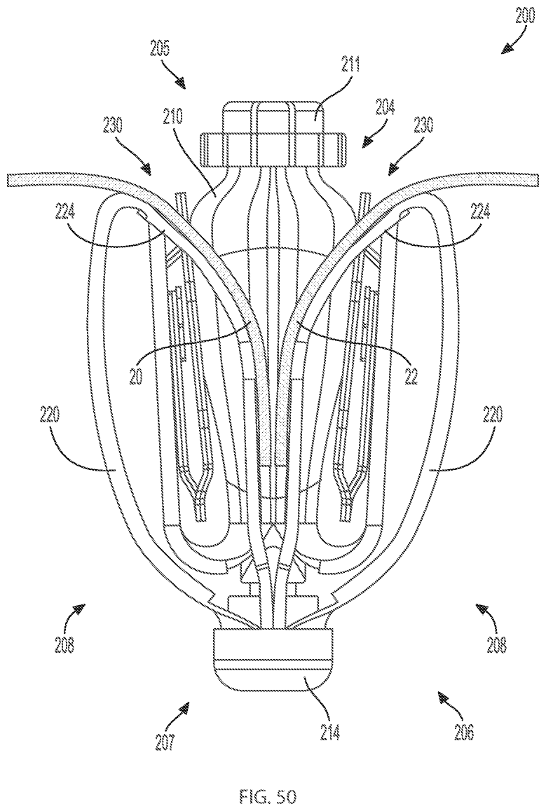

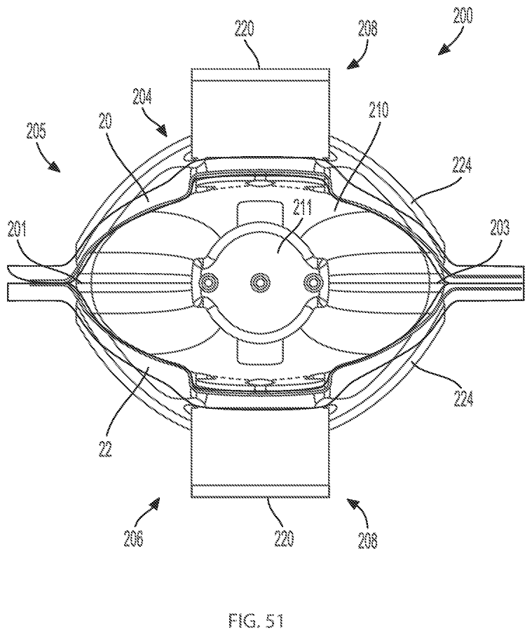

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20, 22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

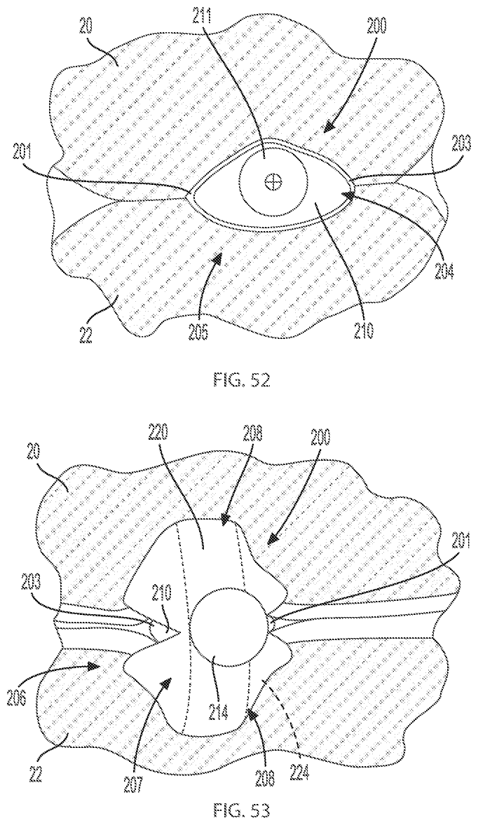

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or inhibited. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

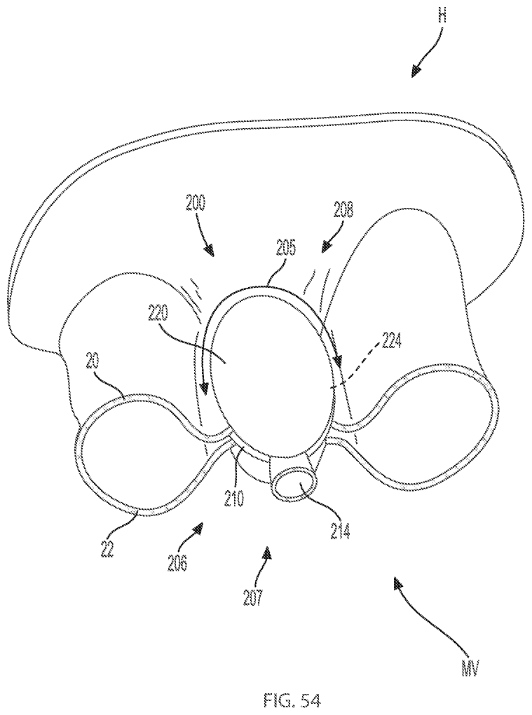

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial— lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area. For example, in FIG. 54, the force on the leaflets 20, 22 by the paddle frames is spread along the entire rounded length of the paddle frame 224, as the leaflets 20 try to open during the diastole cycle.

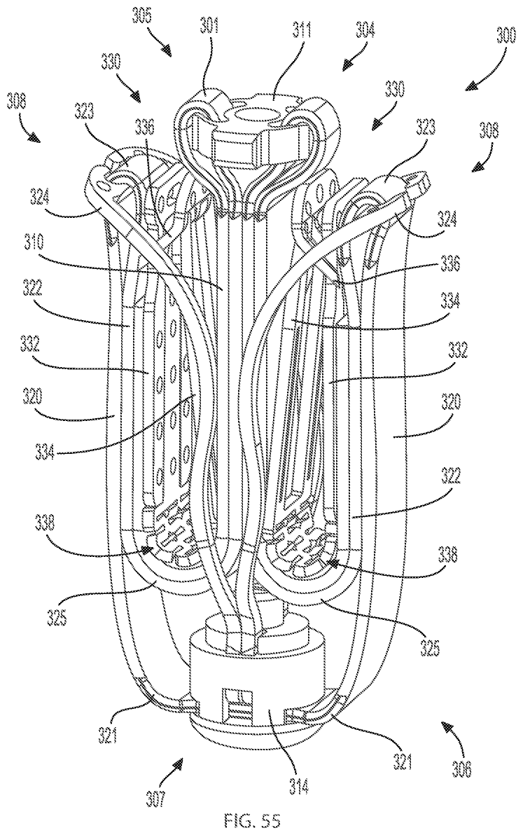

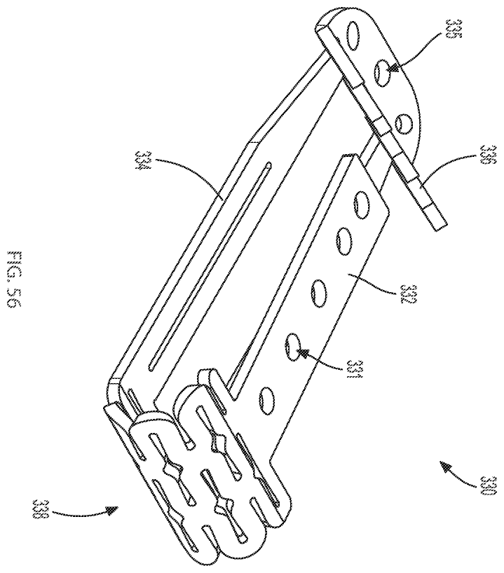

Referring now to FIG. 55, an example of an implantable prosthetic device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. For example, as shown in the illustrated implementation, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from the continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.) and thus the anchors 308 move relative to a midpoint of the device. This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

In some implementations, the clasps comprise a moveable arm coupled to an anchor. In some implementations, the clasps 330 (as shown in detail in FIG. 56) include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines (e.g., the actuation lines 216 shown in FIGS. 43-48) attached to holes 335 in the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

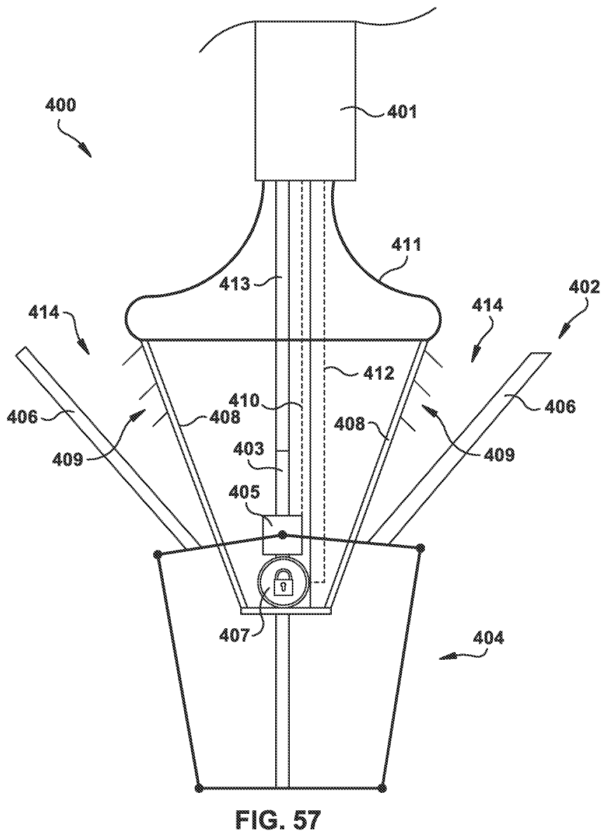

The concepts disclosed by the present application can be used with a wide variety of different valve repair devices. FIGS. 57-63 illustrate another example of one of the many valve repair systems 400 for repairing a native valve of a patient that the concepts of the present application can be applied to. The valve repair system 400 includes a delivery device 401 and a valve repair device 402.

The valve repair device 402 includes a base assembly 404, a pair of paddles 406, and a pair of gripping members 408. In some implementations, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include a barbed portion 409 for attaching the gripping members to valve tissue when the valve repair device 402 is attached to the valve tissue. The gripping member 408 forms a means for gripping the valve tissue (in particular tissue of the valve leaflets) with a sticking means or portion such as the optional barbed portion 409. When the paddles 406 are in the closed position, the paddles engage the gripping members 408, such that, when valve tissue is attached to the optional barbed portion 409 of the gripping members, the paddles act as holding or securing means to hold the valve tissue at the gripping members and to secure the valve repair device 402 to the valve tissue. In some implementations, the gripping members 408 are configured to engage the paddles 406 such that the optional barbed portion 409 engages the valve tissue member and the paddles 406 to secure the valve repair device 402 to the valve tissue member. For example, in certain situations, it can be advantageous to have the paddles 406 maintain an open position and have the gripping members 408 move outward toward the paddles 406 to engage valve tissue and the paddles 406.

While the examples shown in FIGS. 57-63 illustrate a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, such as, for example, a shaft or rod. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, etc.

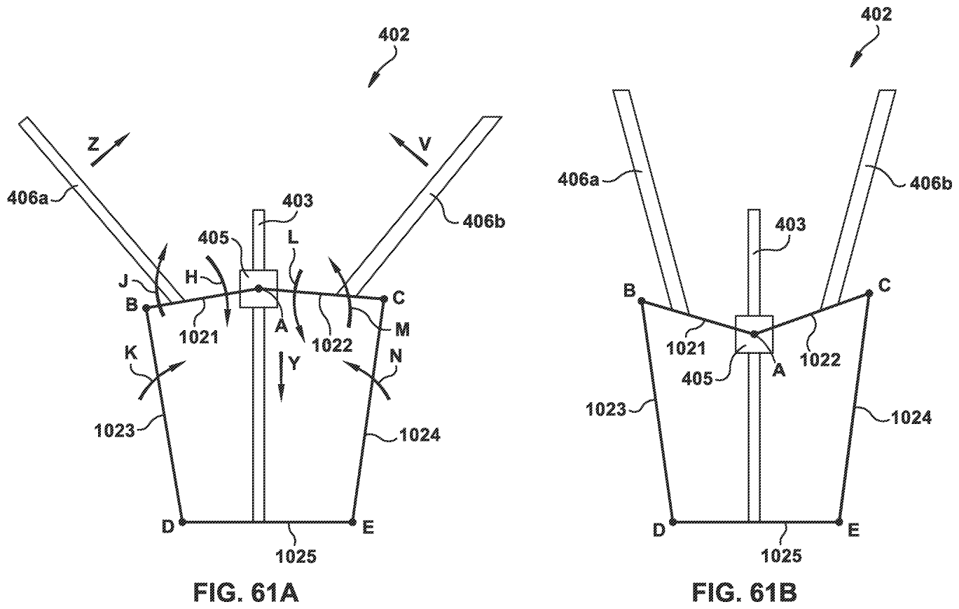

The lock control mechanism 412 is configured to lock and unlock the lock. The lock 407 serves as a locking means for locking the coupler 405 in a stationary position with respect to the shaft 403 and can take a wide variety of different forms and the type of lock control mechanism 412 can be dictated by the type of lock used. In one example, the lock 407 includes a pivotable plate having a hole, in which the shaft 403 of the valve repair device 402 is disposed within the hole of the pivotable plate. In this example, when the pivotable plate is in the tilted position, the pivotable plate engages the shaft 403 to maintain a position on the shaft 403, but, when the pivotable plate is in a substantially non-tilted position, the pivotable plate can be moved along the shaft (which allows the coupler 405 to move along the shaft 403). In other words, the coupler 405 is prevented from moving in the direction Y (as shown in FIG. 61A) along the shaft 403 when the pivotable plate of the lock 407 is in a tilted (or locked) position, and the coupler is allowed to move in the direction Y along the shaft 403 when the pivotable plate is in a substantially non-tilted (or unlocked) position. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position. In some implementations, the pivotable plate of the lock 407 is biased in the tilted (or locked) position, and the lock control mechanism 412 is used to move the plate from the tilted position to the substantially non-tilted (or unlocked) position. In some implementations, the pivotable plate of the lock 407 is biased in the substantially non-tilted (or unlocked) position, and the lock control mechanism 412 is used to move the plate from the substantially non-tilted position to the tilted (or locked) position.

FIGS. 61A-61B illustrate the valve repair device 402 moving from an open position (as shown in FIG. 61A) to a closed position (as shown in FIG. 61B). The base assembly 404 includes a first link 1021 extending from point A to point B, a second link 1022 extending from point A to point C, a third link 1023 extending from point B to point D, a fourth link 1024 extending from point C to point E, and a fifth link 1025 extending from point D to point E. The coupler 405 is movably attached to the shaft 403, and the shaft 403 is fixed to the fifth link 1025. The first link 1021 and the second link 1022 are pivotally attached to the coupler 405 at point A, such that movement of the coupler 405 along the shaft 403 moves the location of point A and, consequently, moves the first link 1021 and the second link 1022. The first link 1021 and the third link 1023 are pivotally attached to each other at point B, and the second link 1022 and the fourth link 1024 are pivotally attached to each other at point C. One paddle 406a is attached to first link 1021 such that movement of first link 1021 causes the paddle 406a to move, and the other paddle 406b is attached to the second link 1022 such that movement of the second link 1022 causes the paddle 406b to move. In some implementations, the paddles 406a, 406b can be connected to links 1023, 1024 or be extensions of links 1023, 1024.

In order to move the valve repair device from the open position (as shown in FIG. 61A) to the closed position (as shown in FIG. 61B), the coupler 405 is moved along the shaft 403 in the direction Y, which moves the pivot point A for the first links 1021 and the second link 1022 to a new position. Movement of the coupler 405 (and pivot point A) in the direction Y causes a portion of the first link 1021 near point A to move in the direction H, and the portion of the first link 1021 near point B to move in the direction J. The paddle 406a is attached to the first link 1021 such that movement of the coupler 405 in the direction Y causes the paddle 406a to move in the direction Z. In addition, the third link 1023 is pivotally attached to the first link 1021 at point B such that movement of the coupler 405 in the direction Y causes the third link 1023 to move in the direction K. Similarly, movement of the coupler 405 (and pivot point A) in the direction Y causes a portion of the second link 1022 near point A to move in the direction L, and the portion of the second link 1022 near point C to move in the direction M. The paddle 406b is attached to the second link 1022 such that movement of the coupler 405 in the direction Y causes the paddle 406b to move in the direction V. In addition, the fourth link 1024 is pivotally attached to the second link 1022 at point C such that movement of the coupler 405 in the direction Y causes the fourth link 1024 to move in the direction N. FIG. 61B illustrates the final position of the valve repair device 402 after the coupler 405 is moved as shown in FIG. 61A.

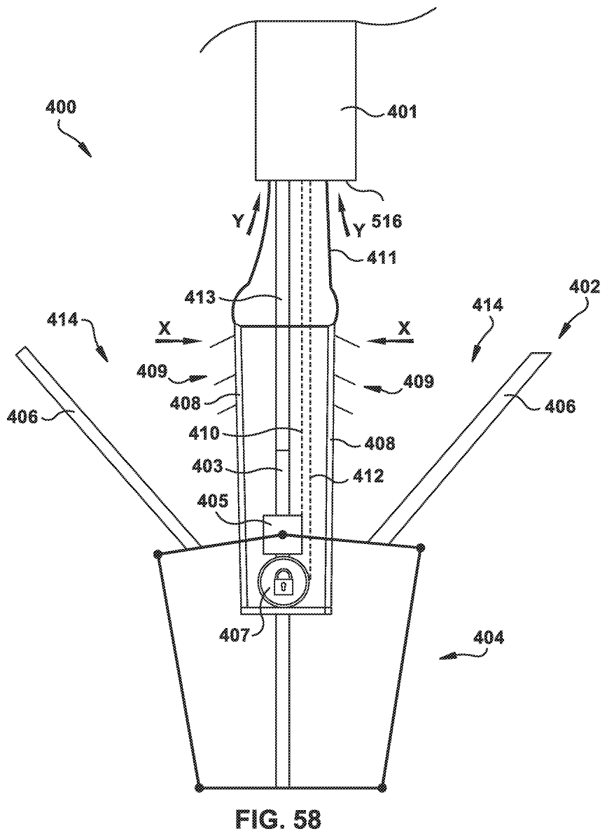

Referring to FIG. 58, the valve repair device 402 is shown in the open position (similar to the position shown in FIG. 61A), and the gripper control mechanism 411 is shown moving the gripping members 408 to provide a wider gap at the opening 414 between the gripping members and the paddles 406. In the illustrated example, the gripper control mechanism 411 includes a line, such as a suture, a wire, etc. that is threaded through an opening in an end of the gripper members 408. Both ends of the line extend through the delivery opening 516 of the delivery device 401. When the line is pulled through the delivery opening 516 in the direction Y, the gripping members 408 move inward in the direction X, which causes the opening 414 between the gripping members and the paddles 406 to become wider.

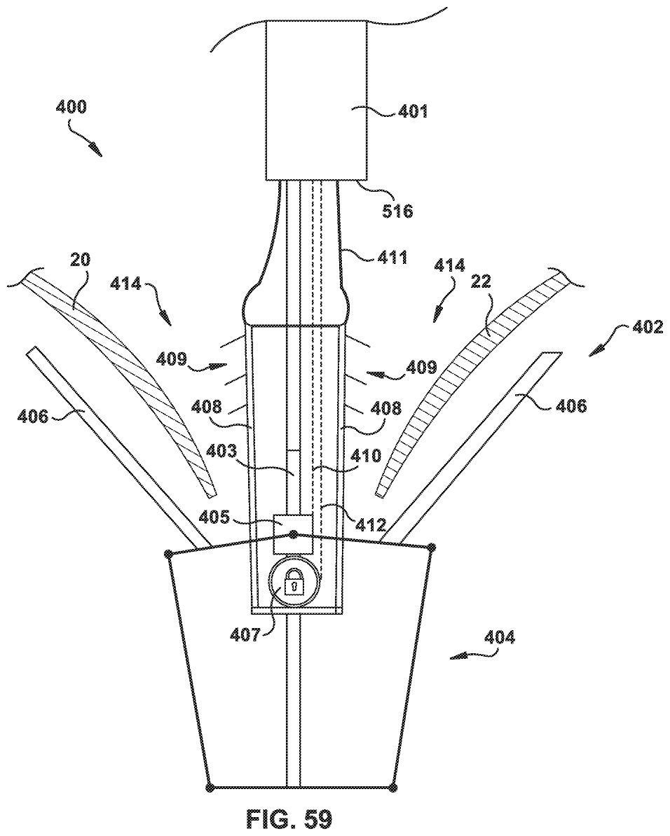

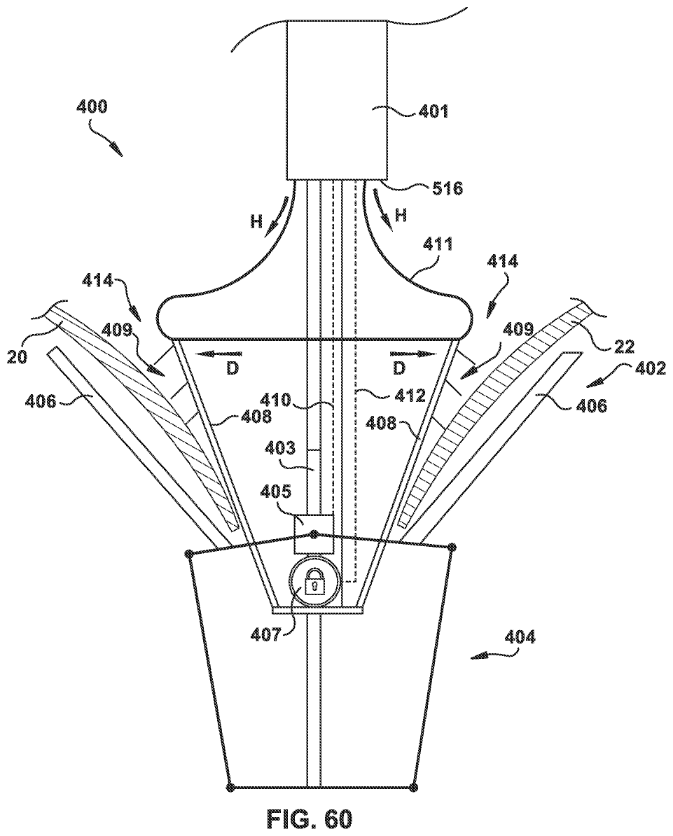

Referring to FIG. 59, the valve repair device 402 is shown such that valve tissue 20, 22 is disposed in the opening 414 between the gripping members 408 and the paddles 406. Referring to FIG. 60, after the valve tissue 20, 22 is disposed between the gripping members 408 and the paddles 406, the gripper control mechanism 411 is used to lessen the width of the opening 414 between the gripping members and the paddles. That is, in the illustrated example, the line of the gripper control mechanism 411 is released from or pushed out of the opening 516 of the delivery member in the direction H, which allows the gripping members 408 to move in the direction D to lessen the width of the opening 414. While the gripper control mechanism 411 is shown moving the gripping members 408 to increase the width of the opening 414 between the gripping members and the paddles 406 (FIG. 59), it should be understood that the gripping members may not need to be moved in order to position valve tissue in the opening 414. In certain circumstances, however, the opening 414 between the paddles 406 and the gripping members 408 can be wider in order to receive the valve tissue.

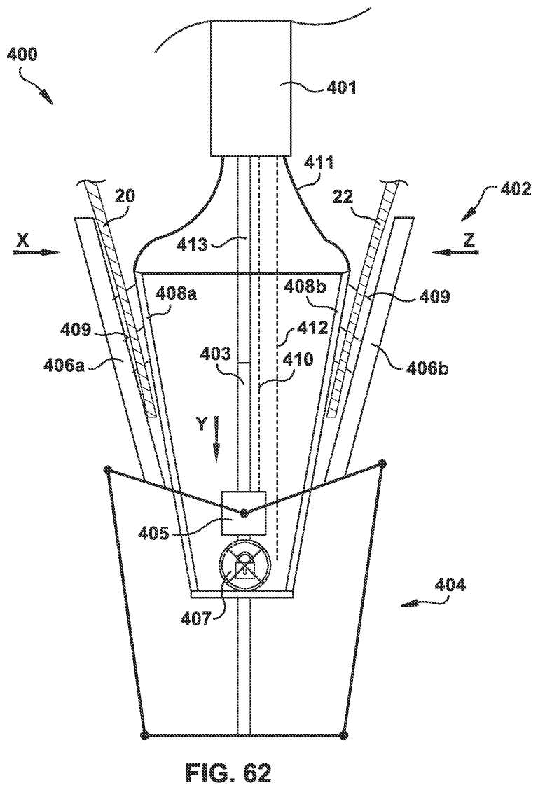

Referring to FIG. 62, the valve repair device 402 is in the closed position and secured to valve tissue 20, 22. The valve repair device 402 is secured to the valve tissue 20 by the paddles 406a, 406b and the gripping members 408a, 408b. In particular, the valve tissue 20, 22 is attached to the valve repair device 402 by the optional barbed portion 409 of the gripping members 408a, 408b, and the paddles 406a, 406b engage the gripping members 408 to secure the valve repair device 402 to the valve tissue 20, 22.

In order to move the valve repair device 402 from the open position to the closed position, the lock 407 is moved to an unlocked condition (as shown in FIG. 62) by the lock control mechanism 412. Once the lock 407 is in the unlocked condition, the coupler 405 can be moved along the shaft 403 by the paddle control mechanism 410. In the illustrated example, the paddle control mechanism 410 moves the coupler 405 in a direction Y along the shaft, which causes one paddle 406a to move in a direction X and the other paddle 406b to move in a direction Z. The movement of the paddles 406a, 406b in the direction X and the direction Z, causes the paddles to engage the gripping members 408a, 408b and secure the valve repair device 402 to the valve tissue 20, 22.

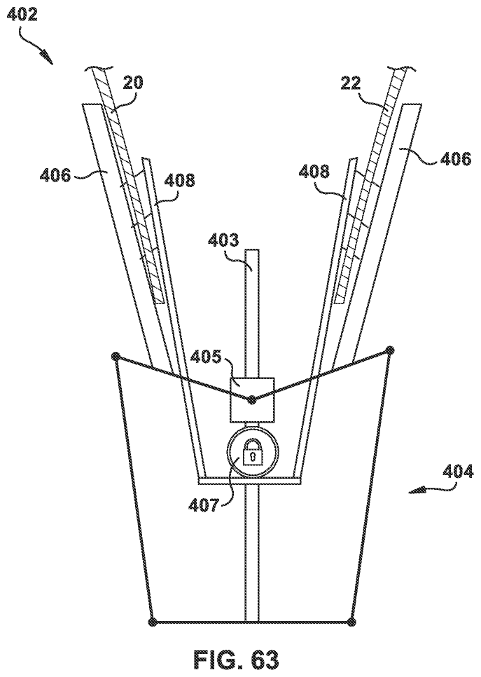

Referring to FIG. 63, after the paddles 406 are moved to the closed position to secure the valve repair device 402 to the valve tissue 20, 22 (as shown in FIG. 62), the lock 407 is moved to the locked condition by the locking control mechanism 412 (FIG. 62) to maintain the valve repair device 402 in the closed position. After the valve repair device 402 is maintained in the locked condition by the lock 407, the valve repair device 402 is removed from the delivery device 401 by disconnecting the shaft 403 from the placement shaft 413 (FIG. 62). In addition, the valve repair device 402 is disengaged from the paddle control mechanism 410 (FIG. 62), the gripper control mechanism 411 (FIG. 62), and the lock control mechanism 412. Removal of the valve repair device 402 from the delivery device 401 allows the valve repair device to remain secured to valve tissue 20, 22 while the delivery device 401 is removed from a patient.

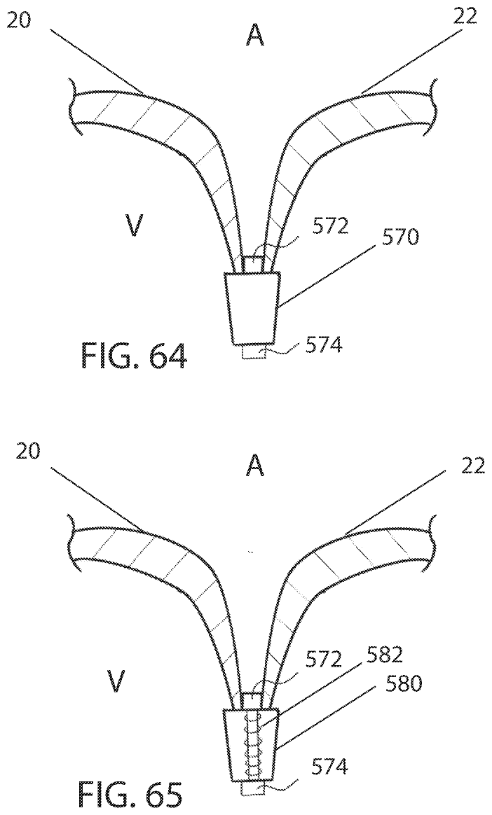

Referring now to FIG. 64, an example valve repair device 570 is shown. The valve repair device 570 can comprise any combination of features of the implantable prosthetic device(s) or implant(s) as described herein. As shown in this example, the valve repair device 570 is deployed between an Atrium A and a Ventricle V, such as in the mitral valve or tricuspid valve of the heart. Valve repair device 570 is engaged with tissue, such as native valve leaflets 20 and 22 to repair the native valve function (e.g., control one-way blood flow from the Atrium A to Ventricle V). As described herein. The valve repair device 570 can be secured in place by paddles, clasps, barbs, anchors, or the like, for example, in any of the manners described herein.

In some implementations, the valve repair device 570 includes one or more sensors, for example, sensor 572 and/or sensor 574. In some implementations, sensor(s) 572 and/or 574 are pressure sensors operable to measure pressures (e.g., blood pressures) proximate to the sensor(s). For example, in one example, the sensor 572 is configured to measure a proximal pressure (i.e., the pressure in the atrium) and sensor 574 is configured to measure a distal pressure (i.e., pressure in the ventricle). Using the measured proximal (atrial) and distal (ventricular) pressures, it is possible to calculate a pressure gradient which offers insight as to the function of the valve repair device and the status of the device within the patient. While sensor(s) are described herein primarily relate to pressure, in some examples the one or more sensors can be configured to measure, collect, interpret, and/or transmit data related and unrelated to pressure, such as, for example, heart rate, physical activity, blood flow, pressure gradient, etc. Furthermore, the ability to observe and collect the above mentioned data in real-time or near-real time enables doctors or other medical professionals to quickly determine the operational effectiveness of the valve repair device.

Some sensor(s) as described herein can be configured to measure, collect, interpret, and/or transmit multiple types of data within a single sensor device. It is appreciated that different sensors are contemplated, such as, for example, pressure plate sensors, capacitive-based sensors, inductive-based sensors, etc. The sensors 572, 574 can be the same type of sensor or can be different types of sensors. It is further appreciated that in some implementations, the sensor(s) 572 and 574 can be embodied in a single sensor configuration. Other configurations, including those with a plurality of sensors are contemplated. With regard to location of sensor(s) 572 and 574, it is appreciated that while depicted in the various locations described herein, the sensor(s) 572 and 574 can, in some implementations, be disposed anywhere on or near a valve repair device.

The sensor(s) 572 and 574 can optionally include a transmitter for wirelessly transmitting data measured by the sensor(s) 572 and 574 in real-time or near real-time. As shown in FIG. 65, an example valve repair device 580 is shown with sensor(s) 572 and 574 and a transmitter 582. The transmitter 582 can take a wide variety of different forms. The transmitter 582 can be an antenna. Such an antenna can take a wide variety of different forms. In the illustrated example, the antenna extends between the sensors 572, 574. In some implementations, the transmitter 582 is a radio-frequency (RF) transmitter. In some implementations, the transmitter 582 is a wi-fi transmitter. In some implementations, the transmitter 582 is a Bluetooth transmitter.

As data is measured, collected, and/or interpreted by the sensor(s) 572 and 574 it can be transmitted wirelessly outside of the body to a compatible receiver device. It is appreciated that the receiver device can be embodied in various devices, including but not limited to, a cell phone, laptop/desktop computer, tablet computer, smart watch, or the like. It is further appreciated that a compatible receiver device can comprise a processor and memory operable to perform calculations, display data, etc. based on the data received from the sensor(s) 572 and 574. In some implementations, the transmitter 582 is configured to transmit and receive data at the sensor(s) 572 and 574. For example, in some implementations, the receiver device is operable to configure and/or calibrate the sensor(s) 572 and 574 via wireless communication with the transmitter 582. It is appreciated that the transmitter 582 as described above can be integrated within the sensor(s) 572 and 574, the valve repair device 580, or both.

In some implementations, the sensor(s) 572 and 574 can include a processor and a memory. The processor and memory configuration can be associated with the sensor(s) and utilized to make various calculations related to the measurements at the sensor(s) 572 and 574. In certain configurations, the sensor(s) 572 and 574 can be further associated with a memory configured to store measured data which can then be used by a processor and/or additional memories to process calculations related to the data. It is appreciated that the processor and memory as described above can be integrated within the sensor(s) 572 and 574, the valve repair device (e.g., valve repair device 570 and/or 580), or both.

In some implementations, the sensor(s) 572 and 574 are battery powered. In some implementations, the sensor(s) 572 and 574 are configured to receive power wirelessly, for example, through a near-field RF power signal. In some implementations, the sensor(s) 572 and 574 would be operable when in communication range with a near-field RF power signal. In some implementations, an example receiver device can transmit such a power signal to the sensor(s) 572 and 574 in order to activate the sensors and facilitate transmission of data from the sensor(s) to the receiver device.

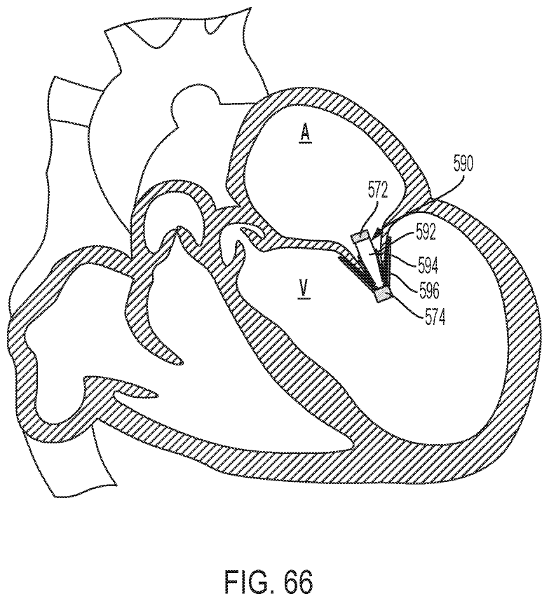

FIG. 66 illustrates an example valve repair device 590 with a spacer 592. The valve repair device 590 can take a wide variety of different forms. For example, the valve repair device 590 can be the valve repair device 100 shown in FIGS. 8-21 and described herein. The illustrated valve repair device 590 includes clasp(s) 594, and paddle(s) 596. The spacer 592, clasp(s) 594, and paddle(s) 596 are used to position and secure the valve repair device 590 in the native valve (e.g., mitral valve, tricuspid valve, etc.) to improve, repair, and/or replace native valve functionality. However, in some examples, the valve repair device 590 can be used in other valves, such as the tricuspid valve, the aortic valve, or the pulmonary valve.

In the example illustrated by FIG. 66, the valve repair device 590 also includes sensor(s) 572 and 574. The spacer 592, clasp(s) 594, and/or paddle(s) 596 can be modified from those of the device 100 to facilitate the inclusion of the sensor(s) 572 and 574. As shown, the sensor 572 can be configured to determine a characteristic or property in the atrium A, such as the pressure in atrium A and the sensor 574 can be configured to determine a characteristic or property in the ventricle, such as the pressure in ventricle V.

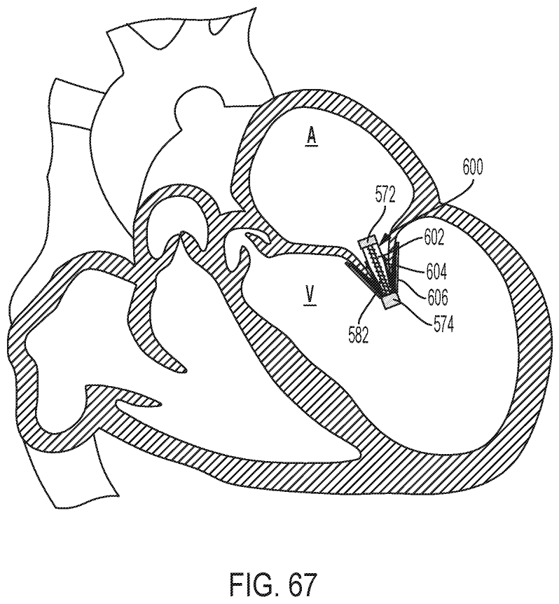

FIG. 67 illustrates an example valve repair device 600. The valve repair device 600 can take a wide variety of different forms. For example, the valve repair device 600 can be the valve repair device 100 shown in FIGS. 8-21 and described herein. The valve repair device 600 can include a coaptation element or spacer 602, clasp(s) 604, and paddle(s) 606. As described herein, coaptation element/spacer 602, clasp(s) 604, and paddle(s) 606 can be used to position and secure the valve repair device 600 in the native valve (e.g., mitral valve, tricuspid valve, etc.) to improve, repair, and/or replace native valve functionality. Also illustrated in FIG. 67 are the sensor(s) 572 and 574 and a transmitter 582. The coaptation element/spacer 602, clasp(s) 604, and/or paddle(s) 606 can be modified from those of the device 100 to facilitate the inclusion of the sensor(s) 572 and 574 and/or the transmitter 582. As shown, the sensor 572 can be configured to determine a proximal pressure in atrium A and the sensor 574 can be configured to determine a distal pressure in ventricle V. The proximal pressure and distal pressure can then be transmitted to a receiving device (not shown) via the transmitter 582.

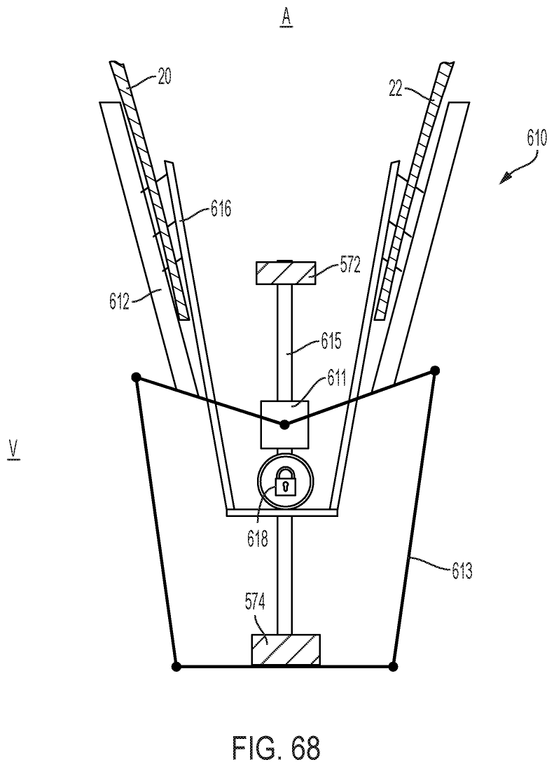

FIG. 68 illustrates an example valve repair device 610 attached to native valve leaflets 20 and 22. The valve repair device 610 can take a wide variety of different forms. For example, the valve repair device 610 can be the valve repair device 402 shown in FIGS. 57-63 and described herein. The valve repair device 610 comprises clasp(s) 616, and paddle(s) 612 that are used to secure the valve repair device 600 in the native valve to repair native valve functionality. The valve repair device 610 includes a linkage 613 that moves the paddles 612. The linkage 613 can be manipulated through movement of a coupler 611 up and down a shaft 615. Once the desired position of the paddles 612 is attained, the coupler can be fixed in place by a lock 618. Also illustrated in FIG. 68 are the sensor(s) 572 and 574. The paddles 612, clasp(s) 616, linkage 613, coupler and/or lock 618 can be modified from those of the device 402 to facilitate the inclusion of the sensor(s) 572 and 574. As shown, the sensor 572 can be configured to determine a proximal pressure in atrium A and the sensor 574 can be configured to determine a distal pressure in ventricle V.

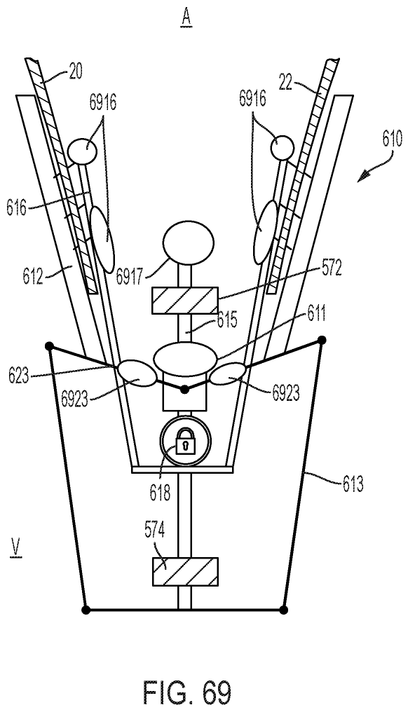

FIG. 69 illustrates that the atrial sensor(s) 572 of the device 610 can be arranged at a wide variety of different positions, including, but not limited to the positions 6916, 6917, and/or 6923. The positions 6916 illustrate that the atrial sensor(s) 572 of the device 610 can be positioned on one or more of the clasps, such as at an end of one or more of the clasps 616 or along the length of one or more of the clasps. The position 6917 illustrates that the atrial sensor(s) 572 of the device 610 can be positioned on the shaft 615, such as at an end of the shafts or along the length of the shaft. The positions 6923 illustrate that the atrial sensor(s) 572 of the device 610 can be positioned at or more positions on the links 623 that are exposed to the atrial pressure.

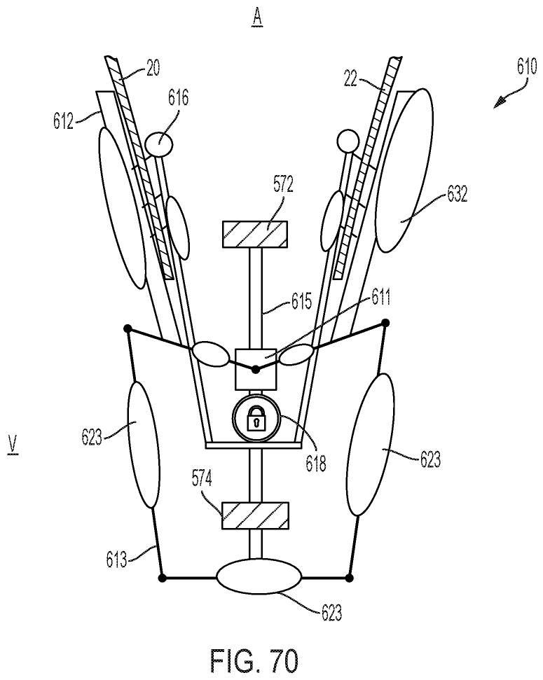

FIG. 70 illustrates that the ventricular sensor(s) 574 of the device 610 can be arranged at a wide variety of different positions, including, but not limited to the positions 623 and the positions 632. The positions 632 illustrate that the ventricle sensor(s) 574 of the device 610 can be positioned on one or more portions of links of the linkage 613 that are exposed to the ventricular pressure. The positions 632 illustrate that the ventricular sensor(s) 574 of the device 610 can be positioned on one or more portions of the paddles 612.

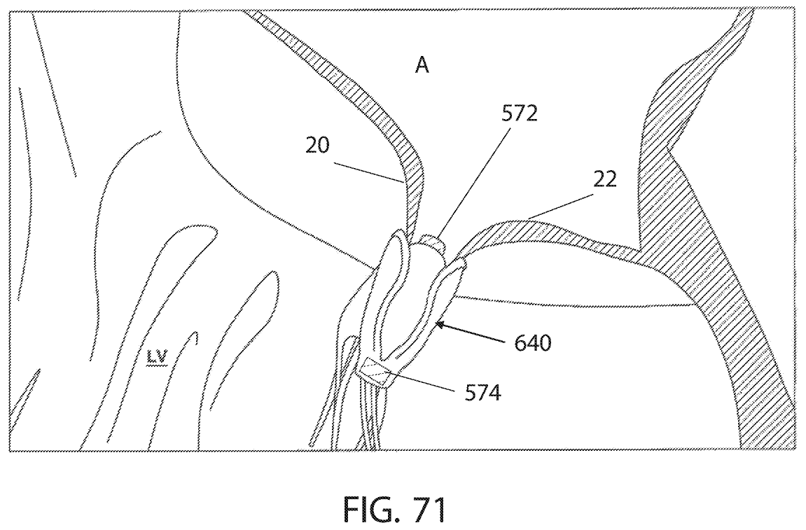

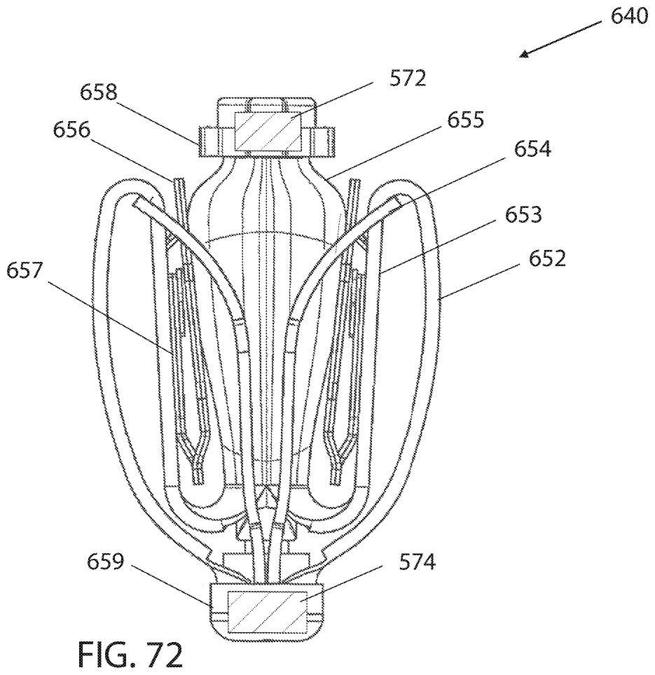

FIGS. 71 and 72 illustrate an example valve repair device 640. The valve repair device 640 can take a wide variety of different forms. For example, the valve repair device 640 can be the valve repair device 200 shown in FIGS. 22-53 and described herein. The valve repair device 640 further comprises outer paddle(s) 652, inner paddle(s) 653, paddle frame 654, a spacer 655, moveable clasp arm(s) 656, and fixed clasp arm(s) 657. The paddle(s) and clasp(s) are used to position and secure the valve repair device 640 in the native valve to repair native valve functionality. The valve repair device 640 can further comprise a collar 658 and a cap 659. Also illustrated in FIG. 72 are the sensor(s) 572 and 574. The outer paddle(s) 652, inner paddle(s) 653, paddle frame 654, spacer 655, moveable clasp arm(s) 656, fixed clasp arm(s) 657, collar 658 and/or the cap 659 can be modified from those of the device 402 to facilitate the inclusion of the sensor(s) 572 and 574 and/or a transmitter 582. As shown, the sensor 572 can be configured to determine a proximal pressure in atrium A and the sensor 574 can be configured to determine a distal pressure in ventricle V.

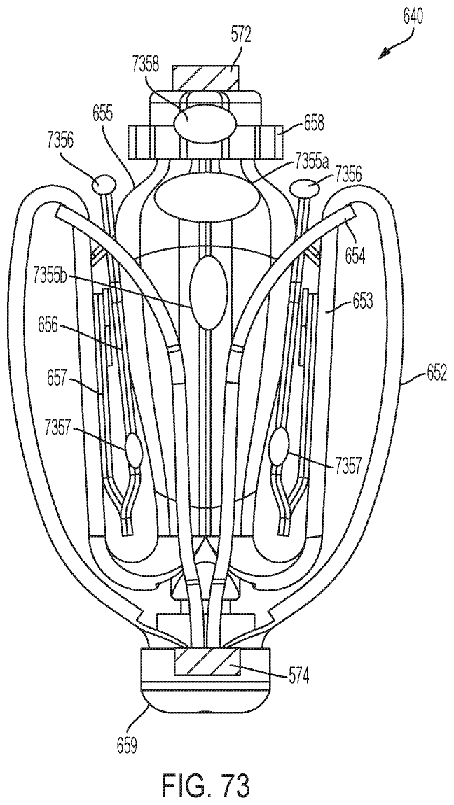

FIG. 73 illustrates that the atrial sensor(s) 572 of the device 640 can be arranged at a wide variety of different positions, including, but not limited to the positions 7358, 7355a, 7355b, 7356, and/or 7357. The position 7358 illustrates that the atrial sensor(s) 572 of the device 640 can be positioned on the collar 658. The positions 7355a, 7355b illustrate that the sensor(s) can be positioned on the spacer 655. The position 7355a illustrates that the sensor(s) can be positioned on a proximal end of the spacer 655. The position 7355b illustrates that the sensor(s) can be positioned on a middle portion along the length of the coaptation element/spacer 655. The positions 7356, 7357 illustrate that the sensor(s) can be positioned on the moveable clasp arm(s) 656. The position 7356 illustrates that the sensor(s) can be positioned on an end of the moveable clasp arm(s) 656. The position 7357 illustrates that the sensor(s) can be positioned along the length of the moveable clasp arm(s) 656.

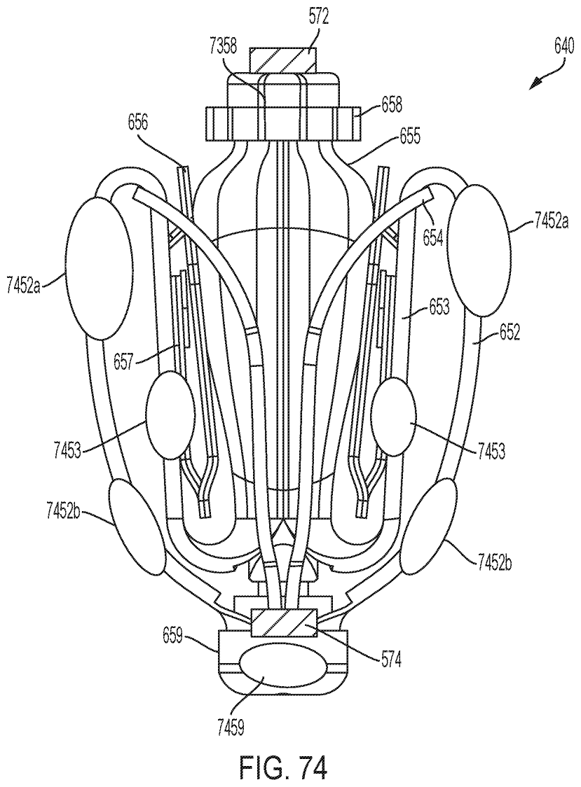

FIG. 74 illustrates that the ventricular sensor(s) 574 of the device 640 can be arranged at a wide variety of different positions, including, but not limited to the positions 7452a, 7452b, 7453, and 7459. The positions 7452a, 7452b illustrate that the ventricle sensor(s) 574 of the device 640 can be positioned on one or more portions of the outer paddles 652 that are exposed to the ventricular pressure. The positions 7452a illustrate that the ventricular sensor(s) 574 of the device 640 can be positioned on one or more proximal portions of the outer paddles 652. The positions 7452b illustrate that the ventricular sensor(s) 574 of the device 640 can be positioned on one or more distal portions of the outer paddles 652. The positions 7453 illustrate that the ventricular sensor(s) 574 of the device 640 can be positioned on one or more portions of the inner paddles 653 and/or one or more portions of the fixed clasp arms 657. The positions 7459 illustrate that the ventricular sensor(s) 574 of the device 640 can be positioned on the cap 659.

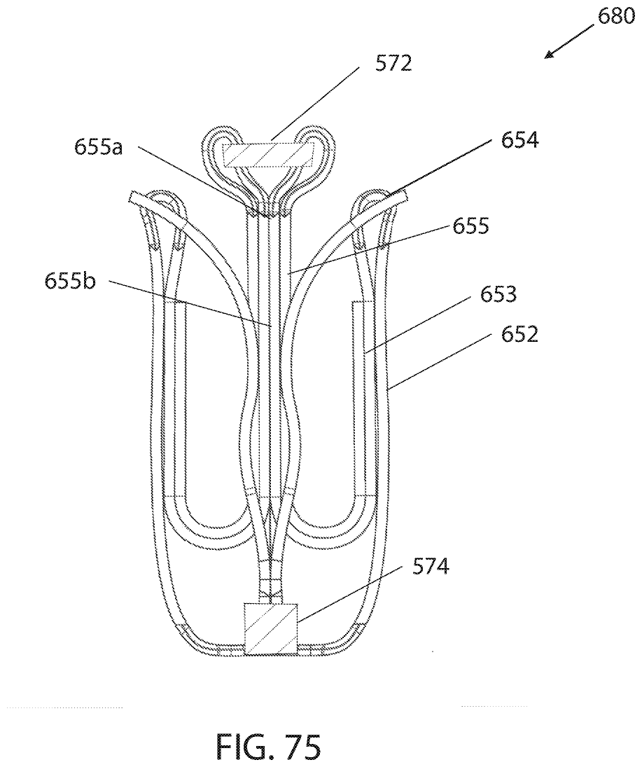

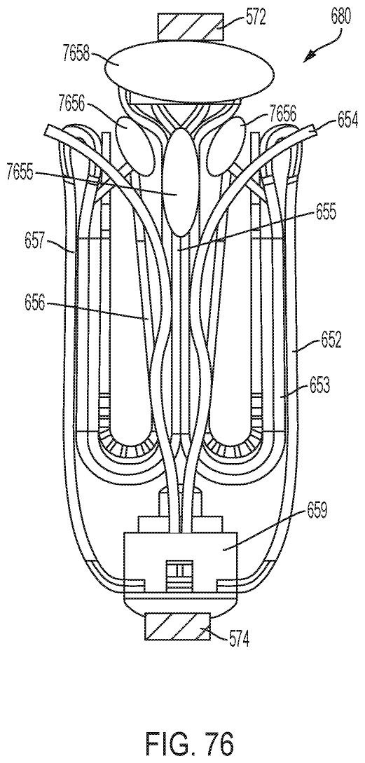

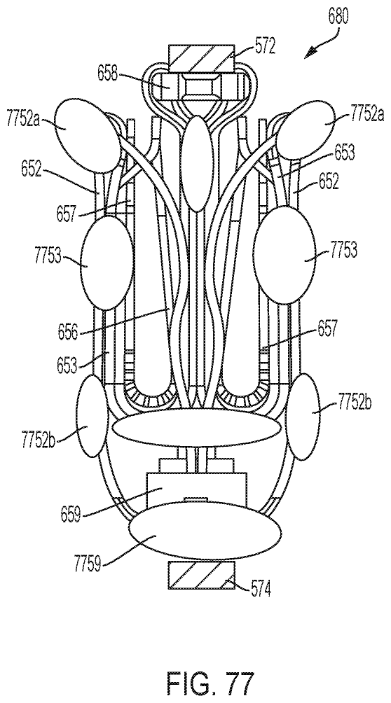

FIG. 75 illustrates an example valve repair device 680. The valve repair device 640 can take a wide variety of different forms. For example, the valve repair device 640 can be the valve repair device 300 shown in FIG. 55 and described herein. The valve repair device 680 comprises outer paddle(s) 652, inner paddle(s) 653, paddle frame 654, spacer 655 (comprising top portion 655a and middle portion 655b), moveable clasp arm(s) 656 (see FIGS. 76 and 77), and fixed clasp arm(s) 657 (see FIGS. 76 and 77). The spacer, paddle(s) and clasp(s) are used to position and secure the valve repair device 680 in the native valve to repair native valve functionality. The valve repair device 680 can also include a collar 658 (FIG. 77) and/or a cap 659 (FIG. 76). Also illustrated in FIG. 75 is the sensor(s) 572 and 574. The outer paddle(s) 652, inner paddle(s) 653, paddle frame 654, spacer 655, moveable clasp arm(s) 656, fixed clasp arm(s) 657, collar 658 and/or the cap 659 can be modified from those of the device 300 to facilitate the inclusion of the sensor(s) 572 and 574 and/or a transmitter 582. As shown, the sensor 572 can be configured to determine a proximal pressure in atrium A and the sensor 574 can be configured to determine a distal pressure in ventricle V.

FIG. 76 illustrates that the atrial sensor(s) 572 of the device 680 can be arranged at a wide variety of different positions, including, but not limited to the positions 7658, 7655, and/or 7456. The position 7658 illustrates that the atrial sensor(s) 572 of the device 640 can be positioned on the collar 658 (see FIG. 77). The position 7655 illustrate that the sensor(s) can be positioned on the spacer 655. The positions 7656 illustrates that the sensor(s) can be positioned on the moveable clasp arm(s) 656.

FIG. 77 illustrates that the ventricular sensor(s) 574 of the device 680 can be arranged at a wide variety of different positions, including, but not limited to the positions 7752a, 7752b, 7753, and 7759. The positions 7752a, 7752b illustrate that the ventricle sensor(s) 574 of the device 640 can be positioned on one or more portions of the outer paddles 652 that are exposed to the ventricular pressure. The positions 7752a illustrate that the ventricular sensor(s) 574 of the device 640 can be positioned on one or more proximal portions of the outer paddles 652. The positions 7752b illustrate that the ventricular sensor(s) 574 of the device 640 can be positioned on one or more distal portions of the outer paddles 652. The positions 7753 illustrate that the ventricular sensor(s) 574 of the device 640 can be positioned on one or more portions of the inner paddles 653 and/or one or more portions of the fixed clasp arms 657. The positions 7759 illustrate that the ventricular sensor(s) 574 of the device 640 can be positioned on the cap 659.

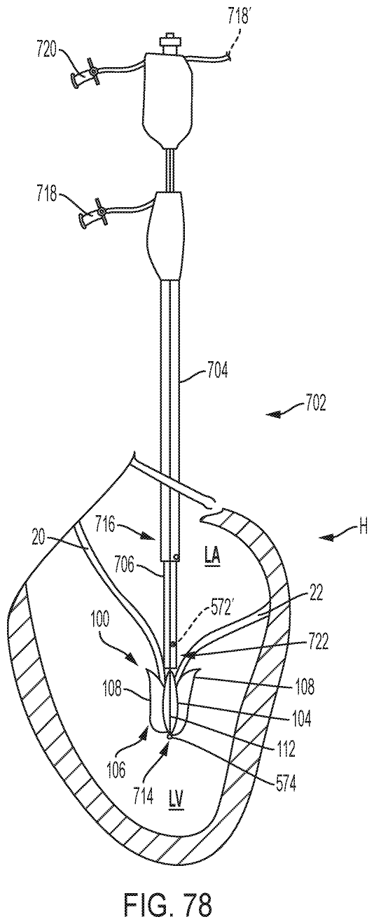

FIG. 78 shows an example delivery system 702 deploying a valve repair device in a human heart H. In the illustrated implementation, as opposed to the valve repair device (e.g., valve repair device 570) including the one or more sensors, for example, sensor 572, and/or sensor 574, the one or more sensors are positioned on one or more components of the delivery system 702. However, in some implementations the sensor 572 or the sensor 574 can be included on the valve repair device in any of the manners disclosed herein and the other sensor can be included on one or more components of the delivery system. The valve repair device can be any of the valve repair devices disclosed herein, for example valve repair device 100.

FIG. 78 illustrates the valve repair device 100 positioned at the mitral valve MV between the left atrium LA and the left ventricle LV and engaging valve tissue 20, 22 as part of any suitable valve repair system (e.g., any valve repair system disclosed in the present application). The delivery system 702 can be configured to position the valve repair device at the mitral valve MV between the left atrium LA and the left ventricle LV in a wide variety of different ways. For example, the valve repair device can be delivered through the atrium as shown, transapically, transeptally, etc. In FIG. 78, the delivery through the atrium is selected merely because it provides the simplest illustration of the system. In addition, the valve repair device 10 can be configured for implanting on other native heart valves, such as the tricuspid valve.

The device or implant 100 includes the coaptation element 110 (e.g., spacer, plug, filler, foam, sheet, membrane, coaption element, etc.) that is adapted to be implanted between the leaflets 20, 22 of a native valve (e.g., a native mitral valve MV, native tricuspid valve, etc.) and is slidably attached to an actuation element 112 (e.g., actuation wire, actuation shaft, actuation tube, etc.). The anchor portion 106 of the device 100 includes one or more anchors 108 that are actuatable between open and closed conditions and can take a wide variety of forms, such as, for example, paddles, gripping elements, or the like. The actuation of actuation element 112 opens and closes the anchor portion 106 of the device 100 to grasp the native valve leaflets 20, 22 during implantation.

In some implementations, the delivery system 702 includes a steerable catheter 704, an implant catheter 706, and an actuation element 112. These can be configured to extend through a guide catheter/sheath (e.g., a transseptal sheath, etc.). In some implementations, the actuation element 112 extends through the implant catheter 706 and the coaptation element 110 to a distal end 714 of the anchor portion 106.

In some implementations, the sensors 572, 574 are pressure sensors operable to measure pressures proximate to the sensors. For example, in one example, the first sensor 572 is configured to measure a proximal pressure (i.e., the pressure in the left atrium) and the second sensor 574 is configured to measure a distal pressure (i.e., pressure in the left ventricle). The first sensor 572 and the second sensor 574 can be located on the delivery system 702 in any suitable location to measure the proximal and distal pressure. Using the measured proximal (atrial) and distal (ventricular) pressures, it is possible to calculate a pressure gradient which offers insight as to the function of the valve repair device and the status of the device within the patient. While sensor(s) are described herein primarily relate to pressure, in some examples the one or more sensors can be configured to measure, collect, interpret, and/or transmit data related and unrelated to pressure, such as, for example, heart rate, physical activity, blood flow, pressure gradient, etc. Furthermore, the ability to observe and collect the above mentioned data in real-time or near-real time enables doctors or other medical professionals to quickly determine the operational effectiveness of the valve repair device.

In some implementations, the first sensor 572 and the second sensor 574 comprise fluid-filled lumens where each lumen forms a continuous fluid path, allows concurrent real-time assessment of atrial and ventricular pressure, and thus, allows for transvalvular gradient assessment. The first sensor 572 and the second sensor 574 can be provided in the delivery system 702 in any suitable location to measure the proximal and distal pressure. In some implementations, the first sensor 572 can be a first lumen formed in the steerable catheter 704 and extending from a distal portion 716 of the steerable catheter 704 to a first outlet pressure port 718 that can be connected to a pressure transducer (not shown) or other pressure sensing device. The fluid (e.g., saline) in the first lumen forms a continuous fluid path that is capable of relaying a pressure signal along the first lumen from the distal portion 716 of the steerable catheter to the pressure transducer so that real-time pressure can be monitored. Since the distal portion 716 of the steerable catheter 704 is positioned in the left atrium LA during deployment of the device or implant 100, the first sensor 572 can measure atrial pressure.

In a similar manner, the second sensor 574 can be a second lumen formed in one or more of the implant catheter 706 and the means for actuating or actuation element 112. For example, the means for actuating or actuation element 112 can be an actuation tube that includes the second lumen or a portion of the second lumen. The tubular actuation element 112 extends through the implant catheter 706 from the distal end 714 of the device or implant 100. The tubular actuation element can be in fluid communication with a second outlet pressure port 720 that can be connected to a pressure transducer (not shown) or other pressure sensing device. The fluid (e.g., saline) in the second lumen forms a continuous fluid path that is capable of relaying a pressure signal along the second lumen from the distal end 714 of the device or implant 100 to the pressure transducer so that real-time pressure can be monitored. Since the distal end 714 of the device or implant 100 is positioned in the left ventricle LV, the second sensor 574 can measure ventricular pressure which can be relayed along the implant catheter 706 and be monitored real-time and simultaneously similarly to atrial pressure. Combining the atrial and ventricular pressure assessment, users can assess transvalvular gradient before and after the implant procedure to evaluate procedural success.

In some implementations, the first lumen and the second lumen can both be formed in the implant catheter 706. For example, the second sensor 574 can comprise the actuation element 112 and a lumen in the implant catheter that is disposed around the actuation element. An optional seal can be provided between the actuation element 112 and the implant catheter 706 that prevents, substantially prevents, or inhibits fluid in the atrium from entering the lumen in the implant catheter that is disposed around the actuation element 112, but allows the actuation element to slide relative to the implant catheter 706. The lumen in the implant catheter that is disposed around the actuation element and the actuation element 112 extend from the distal end 714 of the device or implant 100 and are in communication with a second outlet pressure port 720, to measure ventricular pressure. The first sensor 572′ can be a first lumen, that instead of being formed in the steerable catheter 704, is formed in the implant catheter 706 and extends from a distal portion 722 of the implant catheter 706 to an outlet pressure port 718′ that can be connected to a pressure transducer (not shown) or other pressure sensing device. The distal portion 722 of the implant catheter 706 remains in the left atrium during deployment of the device or implant 100 so that the fluid (e.g., saline) in the first lumen forms a continuous fluid path that is capable of relaying a pressure signal along the first lumen from the distal portion 722 of the implant catheter 706 to the pressure transducer so that real-time pressure can be monitored. Since the distal portion 722 of the implant catheter 706 is positioned in the left atrium LA, the first sensor 572′ can measure atrial pressure.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the various examples, these various aspects, concepts, and features can be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures—such as alternative materials, structures, configurations, methods, devices, and components, alternatives as to form, fit, and function, and so on—can be described herein, such descriptions are not intended to be a complete or exhaustive list of available alternative implementations, whether presently known or later developed. Those skilled in the art can readily adopt one or more of the inventive aspects, concepts, or features into additional implementations and uses within the scope of the present application even if such implementations are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts can be expressly identified herein as being inventive or forming part of a disclosure, such identification is not intended to be exclusive, but rather there can be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the implementations in the specification.

What is claimed is:

1. A sensing valve repair device comprising:
a pair of inner paddles;
a pair of outer paddles connected to the pair of inner paddles;
a pair of clasps, each clasp having a fixed arm attached to one of the pair of inner paddles, a movable arm, and a hinge portion connecting the movable arm to the fixed arm;
a pair of sensors; and
wherein each sensor of the pair of sensors is attached to one or more of one the fixed arms of the pair of clasps and one of the pair of inner paddles.

2. The sensing valve repair device of claim 1 wherein the sensor is configured to sense one or more of pressure, capacitance, and inductance.

3. The sensing valve repair device of claim 1 wherein at least a portion of each sensor is disposed in a space between the movable arm and the fixed arm of one of the pair of clasps.

4. The sensing valve repair device of claim 1 wherein at least a portion of each sensor is closer to the hinge portion than a free end of the movable arm of one of the pair of clasps.

5. The sensing valve repair device of claim 1 further comprising a coaptation element attached to the pair of inner paddles.

6. The sensing valve repair device of claim 1 further comprising a transmitter configured to a transmit sensed data from at least one of the pair sensors to a receiver.

7. The sensing valve repair device of claim 5 further comprising a ventricular pressure sensor disposed at a distal end of the device and an atrial pressure sensor disposed at a proximal end of the device.

8. The sensing valve repair device of claim 1 wherein the sensing valve repair device is configured for implantation within a mitral valve.

9. A sensing valve repair system comprising:
a delivery catheter;
a sensing valve repair device coupled to the delivery catheter, wherein the sensing valve repair device comprises;
a pair of inner paddles;
a pair of outer paddles connected to the pair of inner paddles;
a pair of clasps, each clasp having a fixed arm attached to one of the pair of inner paddles, a movable arm, and a hinge portion connecting the movable arm to the fixed arm;
a pair of sensors; and
wherein each sensor of the pair of sensors is attached to one or more of one the fixed arms of the pair of clasps and one of the pair of inner paddles.

10. The sensing valve repair system of claim 9 wherein the sensor is configured to sense one or more of pressure, capacitance, and inductance.

11. The sensing valve repair system of claim 9 wherein at least a portion of each sensor is disposed in a space between the movable arm and the fixed arm of one of the pair of clasps.

12. The sensing valve repair system of claim 9 wherein at least a portion of each sensor is closer to the hinge portion than a free end of the movable arm of one of the pair of clasps.

13. The sensing valve repair system of claim 9 further comprising a coaptation element attached to the pair of inner paddles.

14. The sensing valve repair system of claim 9 further comprising a transmitter configured to a transmit sensed data from at least one of the pair sensors to a receiver.

15. The sensing valve repair system of claim 14 further comprising a ventricular pressure sensor disposed at a distal end of the device and an atrial pressure sensor disposed at a proximal end of the device.

16. The sensing valve repair system of claim 9 wherein the sensing valve repair device is configured for implantation within a mitral valve.

17. A sensing valve repair system, comprising:
a steerable catheter;
an implant catheter received inside the steerable catheter;
a valve repair device coupled to the implant catheter;
a first sensor associated with one of the steerable catheter and the implant catheter, wherein the first sensor is configured to sense a characteristic proximal to, or at a proximal end of, the valve repair device; and
a second sensor associated with the implant catheter, wherein the second sensor is configured to sense a characteristic distal to, or at a distal end of, the valve repair device.

18. The sensing valve repair system of claim 17, wherein the characteristic sensed by both the first sensor and the second sensor is pressure.

19. The sensing valve repair system of claim 17, wherein the first sensor is a pressure sensor that includes a first lumen of the implant catheter and is in fluid communication with a first pressure sensing device, wherein the first lumen extends from a distal portion of the implant catheter to the first pressure sensing device, wherein the second sensor is a pressure sensor that includes a second lumen of the implant catheter and is in fluid communication with a second pressure sensing device.

20. The sensing valve repair system of claim 17, further comprising a transmitter configured to transmit sensed data from at least one of the first and second sensors to a receiver.
